(19) United States
(12) Patent Application Publication
(10) Pub. No.: US 2005/0261214 A1
(43) Pub. Date: Nov. 24, 2005

(54) Complexes for transferring substances of interest into a cell

(76) Inventors: Serge Braun, Dorlisheim (FR); Olivier Meyer, Montreal Quebec (CA); Abdesslame Nazih, Strasbourg (FR); Denis Heissler, Eckbolsheim (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/476,247

(22) PCT Filed: May 14, 2002
(86) PCT No.: PCT/EP02/05304

§ 371 Date: May 11, 2004

Related U.S. Application Data
(60) Provisional application No. 60/293,188, filed on May 25, 2001.

(30) Foreign Application Priority Data
May 15, 2001 (EP) 01440134.3

(51) Int. Cl. A61K 48/00; C12N 15/85

(52) U.S. Cl. 514/44; 435/455

(57) ABSTRACT

The present invention concerns new polar compounds, complexes and compositions comprising them, wherein said compound comprises: (i) a polar headgroup spacer, (ii) at least one hydrophobic moiety, and (iii) at least one hydrophilic polymer, and wherein said polar headgroup spacer is coupled to said hydrophobic moiety and to said hydrophilic polymer.

The present invention concerns new polar compounds, complexes and compositions comprising them. More particularly, the present invention concerns the use of said compounds or of said compositions to prepare complexes for transferring a substance of interest into a cell. These complexes are useful for delivering said substance into a cell, particularly in gene transfer applications.

Gene therapy has generally been conceived as principally applicable to heritable deficiency diseases (cystic fibrosis, dystrophies, haemophilias, . . . ) where permanent cure or improvement of the patient condition may be effected by introducing a functional gene in cells. However, a much larger group of diseases, notably acquired diseases (cancer, AIDS, multiple sclerosis, . . . ) might be treatable by transiently engineering host cells to produce beneficial proteins. Another application of gene therapy is vaccination. In this regard, the immunogenic product encoded by the nucleic acid introduced into cells of a vertebrate may be expressed and secreted or be presented by said cells in the context of the major histocompatibility antigens, thereby eliciting an immune response against the expressed immunogen. Functional nucleic acid can be introduced into cells by a variety of techniques resulting in either transient expression of the gene of interest, referred to as transient transfection, or permanent transformation of the host cells resulting from incorporation of the nucleic acid into the host genome.

The first clinical protocol applied to man was initiated in the USA in September 1990 on a patient suffering from adenine deaminase (ADA) deficiency. This first encouraging experiment has been followed by numerous new applications, including vaccination, and promising clinical trials based on gene therapy are currently ongoing (see for example clinical trials listed at http://cnetdb.nci.nih.gov/trialsrch.shtmlor http://www.wiley.co.uk/genetherapy/clinical/).

Successful nucleic acid based therapy depends principally on the efficient delivery of a nucleic acid of interest, for example a gene encoding protein, to make its function or expression possible in cells of a living organism. Said genetic material can be transferred into cells using a wide variety of vectors resulting in either transient expression or permanent transformation of the host genome. During the past decade, a large number of viral, as well as non-viral, vectors has been developed for supporting said transfer (see for example Robbins et al., 1998, Tibtech 16, 35-40; Rolland, 1998, Therapeutic Drug Carrier Systems 15, 143-198 for reviews). This transfer is usually referred to nucleic acid delivery.

Most delivery systems used to date are viral vectors, especially adeno-, pox- and retroviral vectors (see Robbins et al., 1998, Tibtech, 16, 35-40 or Walther and Stein, 2000, Drugs, 60, 249-271 for a review). Viruses have developed diverse and highly sophisticated mechanisms to achieve this goal including crossing of the cellular membrane, escape from endosomes and lysosomal degradation, and finally delivery of their genome to the nucleus followed by expression of the viral genome. In consequence, viruses have been used in many nucleic acid delivery applications, for example in vaccination or gene therapy applied to humans. However, said use of viruses suffers from a number of disadvantages: retroviral vectors cannot accommodate large-sized nucleotide sequences (e.g. the dystrophin gene which is around 13 kb), the retroviral genome is integrated into host cell DNA and may thus cause genetic changes in the recipient cell and infectious viral particles can disseminate within the organism or into the environment; adenoviral vectors can induce a strong immune response in treated patients and are lacking specificity when infecting cells (Mc Coy et al, 1995, Human Gene Therapy, 6, 1553-1560; Yang et al., 1996, Immunity, 1, 433-442). Nevertheless, despite these drawbacks, viral vectors are currently the most useful delivery systems because of their efficiency.

Besides, in order to offer safer approach for intracellular nucleic acid delivery, non-viral systems have been proposed. In 1990, Wolff et al. (Science, 247, 1465-1468) have shown that injection of naked RNA or DNA, i.e. nucleic acids without any special delivery system, directly into mouse skeletal muscle results in expression of reporter genes within the muscle cells. Nevertheless, although these results indicate that nucleic acid by itself is capable of crossing the plasma membrane of certain cells in vivo, the efficiency of the transfection actually observed remains very limited due, in particular, to the polyanionic nature of nucleic acids which limits their passage through negatively-charged cell membranes. Additionally, said “naked” technology is still inefficient when the nucleic acid is administered systematically.

In 1989, Felgner et al. (Nature, 337, 387-388) proposed the use of cationic lipids in order to facilitate the introduction of large anionic molecules such as nucleic acids into cells. These cationic lipids are capable of forming complexes (i.e. lipoplexes) with anionic molecules, thus tending to neutralize the negative charges of these molecules allowing them to compact into the complex, and favoring their introduction into the cell. Similarly, other non-viral delivery systems have been developed which are based for example on receptor-mediated mechanisms (Perales et al., 1994, Eur. J. Biochem. 226, 255-266; Wagner et al., 1994, Advanced Drug Delivery Reviews, 14, 113-135), on cationic polymers (forming polyplexes when complexed with anionic molecules) such as polyamidoamine (Haensler et Szoka, 1993, Bioconjugate Chem., 4, 372-379), dendritic polymer (WO 95/24221), polyethylene imine or polypropylene imine (WO 96/02655), polylysine (U.S. Pat. No. 5,595,897 or FR2 719 316), or on improved lipids (Felgner et al., 1989, Nature, 337, 387-388) such as DOTMA (Feigner et al., 1987, PNAS, 84, 7413-7417), DOGS or Transfectam™ (Behr et al., 1989, PNAS, 86, 6982-6986), DMRIE or DORIE (Felgner et al., 1993, Methods 5, 67-75), DC-CHOL (Gao et Huang, 1991, BBRC, 179, 280-285), DOTAP™ (McLachlan et al., 1995, Gene Therapy, 2,674-622), Lipofectamine™ or cationic glycerolipid compounds (see for example EP 901 463 and WO98/37916). These non-viral systems present potential advantages with respect to large-scale production, safety, flexibility in their chemical design, low immunogenicity and capacity to deliver large fragments of nucleic acid.

However, several studies (for example, Mahato et al., 1995, J. Pharm. Sci., 84, 1267-1271, Thierry et al., 1995, PNAS 92, 9742-9746) have shown that the transfer efficiency of the complexed anionic substances of interest into the cells, especially in the case of in vivo transfer, can greatly vary in function of the interaction between the complexes and the cell membranes, the cell type involved, the lipid composition of the cationic components, the size of the complexes formed with the anionic molecules and the ratio of the positive to negative charges of the different components of the complex. Currently, very little is known concerning the mechanisms which enable the interaction of the complexes with the cell membranes and the transfer of the complexes into the cell, and the ongoing research remains highly empirical. More particularly, it has been shown that one major pathway for said non-viral vectors intracellular delivery is internalization into cell vesicles by endocytosis. Endocytosis is the natural process by which eukaryotic cells ingest segments of the plasma membrane in the form of small endocytosis vesicles, i.e. endosomes, entrapping extracellular fluid and molecular material, e.g. nucleic acid molecules. It is thus proposed that non-viral vectors would be internalized into cells by a non-specific process. This lack of any cell or tissue specific transfection could actually reduce the overall transfer efficiency as the administered non-viral vector can disseminate throughout the treated patient, without control, especially when administered systematically in vivo. Additionally, special applications can require that the desired transfer is perfectly controlled and specifically directed towards specific cell or tissue (e.g. in therapeutic targeting of cancer cells, of cardiac cells, of cells involved in immunity, . . . ). Such non disseminating property and/or specificity cannot be reached with the presently available vectors.

Consequently, there is still a need to design non-viral, as well as viral, vectors which are capable to more specifically deliver nucleic acids to targeted or restricted type of cells and/or tissues. With this respect, one first issue is to limit the non-specific transfer of vectors by limiting non-specific interaction with cells and secondly to direct the transfer by providing targeted vectors. The use of targeted vectors, which are able to facilitate interaction with selected target cells or tissues, would limit the vector spread, thus increasing transfer efficacy in the desired target cells/tissues, and thus possible therapeutic effect of the transfered substance of interest. This approach is mainly based on the fact that most of cells and/or tissues, in their natural or diseased status, expresses unique cell/tissue surface markers. For example, endothelial cells in rapidly growing tumors express cell surface proteins not present in quiescent endothelium, i.e. αv integrins (Brooks et al., 1994, Science 264, 569) and receptors for certain angiogenic growth factors (Hanahan, 1997, Science 277, 48). Thus, these cell surface markers may be used as targets to direct the vectors to specific cell type. Compounds able to target cell surface markers are disclosed in literature and may be composed of all or part of sugars, glycol, peptides (e.g. GRP, Gastrin Releasing Peptide), oligonucleotides, lipids (especially those with C2-C22), hormones, vitamins, antigens, antibodies (or fragments thereof), specific membrane receptor ligands, ligands capable of reaction with an anti-ligand, or a combination of said compounds, e.g. galactosyl residues to target the asialoglycoprotein receptor on the surface of hepatocytes.

As above mentioned, most of the non-viral vectors used today consists in complexes between a cationic compound, especially amphiphilic cationic compound, and negatively charged nucleic acids. These complexes have usually a net cationic charge allowing their interaction with the positively charged cell surface. However, this interaction essentially based on electrostatic interactions is non-specific. Thus, it is desirable to reduce this non-specific interaction. One possible solution consists in using complexes further containing hydrophilic moieties which are forming a screen on the surface of the complex, hereby masking its cationic charges and therefore reducing non-specific interaction with the cell surface membrane.

WO 99/58694 discloses lipoplexes which have increased shelf life and high transfection activity in vivo. More precisely, the disclosed vectors are lipoplexes wherein stabilizing agent such as polyethyleneglycol-phosphatidylethanolamine PEG-PE has been incorporated. Experimental data have shown that addition of said stabilizing agent into the lipoplexe further inhibits the non-specific in vivo transfection of cells.

WO 00/32803 discloses cationic synthetic vectors which have been designed in order to limit the vector clearance by the immune system when administered systematically or its loss of tranfecting property because of its adsorpsion on plasmatic proteins. More specifically, said invention concerns a nucleic acid transfer agent comprising a hydrophobic spacer chemically bound to a polycation and at least a hydrophilic substituent.

U.S. Pat. No. 5,013,556 concerns liposome compositions for administering drug via the bloodstream including liposomes enrapping the drug which contain between 1-20 mole percent of an amphipathic lipid derivatized with a polyalkylether, such as phosphatidylethanolamine derivatized with polyethyleneglycol.

The Applicant has now identified new compounds useful in particular for limiting non-specific transfer of substances of interest into cells, in particular in in vivo context of nucleic acid transfer, said compounds being also modifiable or usable for developing targeted non-viral vectors. Thus the present invention provides compounds comprising:
(i) a polar headgroup spacer,
(ii) at least one hydrophobic moiety, and
(iii) at least one hydrophilic polymer,
wherein said polar headgroup spacer is coupled to said hydrophobic moiety and to said hydrophilic polymer.

As used herein, the term “polar headgroup spacer” refers to the charged moiety of the compound of the present invention. While “polar” can invariably refer both to anionic or cationic headgroup spacer, the latter is preferred. More specifically, the cationic character of said polar headgroup spacer can, for example, relate to the presence of group which contains a reactive chemical group, such as an amine, acid, ester, aldehyde, or alcohol group, preferably to the presence of at least one quaternary ammonium group, polyamine moiety, amidinium or guanidinium group, or combinations thereof. Such a cationic headgroup spacer is actually a binding headgroup towards negatively charged substances, preferably it is a binding headgroup able to bind with nucleic acids.

As used herein, the term “hydrophobic moiety” means a fatty acid, fatty alcohol, sterol, or any other hydrophobic molecule capable of distribution into a lipid phase from an aqueous medium. For example, an hydrophobic domain may be a diacylglycerol, a phospholipid, a sterol or a diacylamide derivative. According to a preferred embodiment, said hydrophobic moiety comprises at least one hydrocarbon chain, preferably two. More preferably, said hydrocarbon chain comprises at least one alkyl or alkenyl radicals having 6 to 23 carbon atoms (noted C6-C23), which are linear or branched, or radicals —C(═O)—(C6-C23) alkyl or —C(═O)—(C6-C23) alkenyl, or more particularly —C(═O)—(C12-C20) alkyl or —C(═O)—(C12-C20) alkenyl, which are linear or branched, aryl radicals, cycloalkyl radicals, fluoroalkyl radicals, oxyethylene or oxymethylene groups which are optionally repeated, linear or branched, optionally substituted. Substitution can, for example, reside in cationic functions, such as for example amidinium or guanidinium groups, in C1-C5 alkyl radicals (e.g. methyl, ethyl, propyl, . . . ) or in perfluoroalkyl radical.

According to one special embodiment, “said polar headgroup spacer coupled to said hydrophobic moiety>> refers to a cationic lipid. Cationic lipids are widely described in literature (see for example WO 97/29118, WO 98/08489, WO 98/17693). More specifically, according to the present invention, said cationic lipids contains quaternary ammonium groups (e.g. DOTMA or N-[1-(2,3-dioleyloxyl)propyl]-N,N,N-trimethylammonium (Felgner, PNAS 84 (1987), 7413-7417), DOTAP (McLachlan, Gene Therapy 2 (1995), 674-622), DMRIE, DLRIE, DODAB, . . . ), polycations such as lipopolyamines (DOGS, i.e. dioctadecylamidoglycylspermine or Transfectam™ (Behr, PNAS 86 (1989), 6982-6986), DC-Chol or 3 [N-(N′,N′-dimethylaminoethane)-carbamoyl]cholesterol (Gao, BBRC 179 (1991), 280-285), lipids as disclosed in WO 97/18185), lipids having both quaternary ammonium group and polyamine (e.g. DOSPA), lipids containing amidinium groups (e.g. ADPDE, ADOPE, those described in WO 97/31935). They may further consist in Lipofectin™, DMRIE: 1,2-dimiristyloxypropyl-3-dimethyl-hydroxyethylammonium and DORIE: 1,2-diooleyloxypropyl-3-dimethyl-hydroxyethylammonium (Feigner, Methods 5 (1993), 67-75), Lipofectamine™, spermine or spermidine-cholesterol, Lipofectace™ (for a review see Legendre, Medecine/Science 12 (1996), 1334-1341 or Gao, Gene Therapy 2 (1995), 710-722). The present invention further encompasses compounds comprising “polar headgroup spacer coupled to said hydrophobic moiety>> which are as disclosed in patent applications EP 901463, WO 98/14439, WO 97/19675, WO 97/37966, WO 98/37916 or WO 98/56423 and their isomers.

According to a preferred embodiment, said polar headgroup spacer comprises from 2 to 7 positive charges, more particularly from 3 to 5 positive charges, and preferably 5 positive charges.

According to a preferred embodiment, the compound of the present invention comprises a “polar headgroup spacer coupled to said hydrophobic moiety”, i.e. a cationic lipid, of formula I (see EP 901 463): 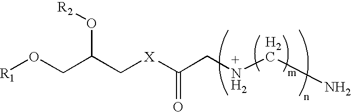
in which:
R₁ and R₂, which are identical or different, are alkyl or alkenyl radicals having 6 to 23 carbon atoms (noted C6-C23), which are linear or branched, or radicals —C(═O)—(C6-C23) alkyl or —C(═O)—(C6-C23) alkenyl, or more particularly —C(═O)—(C12-C20) alkyl or —C(═O)—(C12-C20) alkenyl, which are linear or branched, aryl radicals, cycloalkyl radicals, fluoroalkyl radicals, oxyethylene or oxymethylene groups which are optionally repeated, linear or branched, optionally substituted,
X is an oxygen atom or an amino radical —NR₃, R₃ being a hydrogen atom or an alkyl radical having 1 to 4 carbon atoms,
n is a positive integer from 1 to 6, preferably from 2 to 4,
m is a positive integer from 1 to 6, preferably from 2 to 4, and when n>1, m may be identical or different from said n.

The term “alkenyl” is intended to indicate that the carbon chain in question may comprise one or more double bond(s) along said chain.

In particularly preferred embodiments, the compound of the present invention comprises a cationic lipid selected among the following formulas, respectively numbered II, III, IV, V and VI:

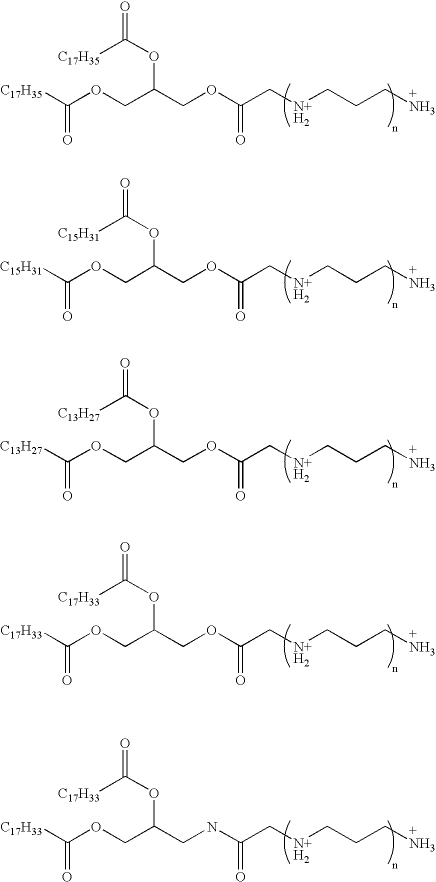

In a particularly preferred embodiment, the compound of the present invention comprises a cationic lipid of formula VI, wherein n is 4. Advantageously, said cationic lipid is further substituted with C1-C5 alkyl radicals (e.g. methyl, ethyl, propyl, . . . ) and/or is perfluorated.

Alternatively, the polar headgroup spacer and hydrophobic moiety are provided in the form of a copolymer including at least one polar polymer and at least one hydrophobic one. Examples of such copolymer are those comprising:
a polar headgroup spacer selected in the group consisting in:
   (i) cationic polymer such as, for example, Poly-L-Lysine, Polyspermine, Poly-N-(2-hydroxypropyl-methacrylamide-b-poly(trimethylammonioethylmethacrylate chloride), N-cetylpyridinium bromide, N-dodecylpyridinium bromide, Polyamidoamine, Polyethylenimine, Spermidine, Protamine, Poly(4-vinylpyridine-stat-N-ethyl-4-vinylpyridinium bromide), Poly(4-vinylpyrimidine-stat-N-ethyl-4-vinylpyridinium bromide-stat-N-cetyl-4-vinylpyridinium bromide, Poly(4-vinylpyrimidinium) or Poly(2-dimethylaminoethylmethacrylate)
   or
   (ii) anionic polymer such as for example Poly(alkylacrylic acid), Poly(N-isopropylacrylamide-methacrylic acid), Poly(lactic acid), Poly(acrylic acid) or Poly(aspartic acid)
      and
an hydrophobic moiety, such as Poly(β-benzyl-L-aspartate), Poly(ε-caprolactone), Polystyrene or Poly(methylmethacrylate), or any of the hydrophobic moities above described.

The skilled man can easily, using his general knowledge, design such copolymers.

The third element of the compound of the invention resides in the hydrophilic polymer (iii). As used herein, the term “hydrophilic polymer” refers to polymers which include, but are not limited to, hydroxy, amino, polyol, sugars (pyranoses or furanoses), or hydrophilic peptides related polymers. The hydrophilic polymer of the invention is preferably selected in the group consisting of polyalkylethers, ganglioside Gm1, polyvinylpyrrolidone, polyalkyloxazoline (e.g. polymethyloxazoline, polyethyloxazoline, polyhydroxypropyloxazoline, . . . ), polyalkylacrylamide (e.g. polyhydroxypropylmethacrylamide, polymethacrylamide, polydimethylacrylamide, . . . ), polyalkylacrylate (e.g. polyhydroxypropylmethacrylate, polyhydroxyethylacrylate, . . . ), polyalkylcellulose (e.g. hydroxymethylcellulose, hydroxyethylcellulose, . . . ), polyaspartamide, tetritols, pentitols, hexitols (i.e. mannitol, sorbitol), dulcitol, . . . According to a more preferred embodiment, the hydrophilic polymer is a polyalkylether, such as for example polyvinylmethylether or polyethyleneglycol (PEG) and related homopolymers, such as polymethylethyleneglycol, polyhydroxypropyleneglycol, polypropyleneglycol, polymethylpropyleneglycol, and polyhydroxypropyleneoxide, or heteropolymers of small alkoxy monomers, such as a polyethetylene/polypropyleneglycol. Advantageously, these polymers have a molecular weight of at least about 120 daltons (Da), and up to about 20,000 daltons (Da). The polyalkylether, such as polyethyleneglycol or polypropyleneglycol, or the methoxy- or ethoxy-capped analogs, can be obtained commercially in a variety of polymer sizes, e.g., 120-20,000 dalton molecular weights. Alternatively, the homo- or heteropolymer can be formed by known polymer synthesis methods to achieve a desired monomeric composition and size. One preferred polyalkylether is PEG, especially those having a molecular weight ranging between about 1,000 and about 5,000 daltons (Da), more preferably of about 2000 Da. The compound of the invention may comprise one or more, similar or different, hydrophilic polymers coupled to the polar headgroup spacer of said molecule.

“Coupled” within the scope of the invention means that the hydrophilic polymer and/or the hydrophobic moiety is covalently or non-covalently linked to said polar headgroup spacer. For sake of clarity, it is stated that the polar headgroup spacer and the hydrophilic polymer are not, in the compounds of the present invention, coupled via the intermediary of any hydrophobic moiety.

“Covalent link” refers to coupling through reactive functional groups, optionally with the intermediary use of a cross linker or other activating agent (see for example Bioconjugate techniques 1996; ed G Hermanson; Academic Press). The polar headgroup spacer (Gao X. et al., Gene Therapy, 1995, 2, 710-722) and/or the hydrophilic polymer and/or the hydrophobic moiety may be modified in order to allow the coupling of the hydrophilic polymer and/or of the hydrophobic moiety via, for example, substitution on an activated carbonyl group (including those activated in situ) or on an imidoester, via addition on an αβ-unsaturated carbonyl group, by reductive amination, nucleophilic substitution on a saturated carbon atom or on a heteroatom, by reaction on aromatic cycles, . . . In particular, coupling may be done using homobifunctional or heterobifunctional cross-linking reagents. Homobifunctional cross linkers including glutaraldehyde, succinic acid and bis-imidoester like DMS (dimethyl suberimidate) can be used to couple amine groups of the polar headgroup spacer to the hydrophilic polymer. Numerous examples are given in Bioconjugate techniques ((1996) 188-228; ed G Hermanson; Academic Press) which are well known by those of the art. Heterobifunctional cross linkers include those having both amine reactive and sulfhydryl-reactive groups, carbonyl-reactive and sulfhydryl-reactive groups and sulfhydryl-reactive groups and photoreactive linkers. Suitable heterobifunctional crosslinkers are, for example, described in Bioconjugate techniques (1996) 229-285; ed G Hermanson; Academic Press) or WO99/40214. Examples are, for example, SPDP (N-succinimidyl 3-(2-pyridyldithio)propionate), SMBP (succinimidyl-4-(p-maleimidophenyl)butyrate), SMPT (succinimidyloxycarbonyl-α-methyl-(α-2-pyridyldithio)toluene), MBS (m-maleimidobenzoyl-N-hydroxysuccinimide ester), SIAB (N-succinimidyl (4 iodoacetyl)aminobenzoate), GMBS (γ-maleimidobutyryloxy)succinimide ester), SIAX (succinimidyl-6-iodoacetyl amino hexonate, SIAC (succinimidyl-4-iodoacetyl amino methyl), NPIA (p-nitrophenyl iodoacetate). Other examples are useful to couple carbohydrate-containing molecules (e.g. env glycoproteins, antibodies) to sulfydryl-reactive groups. Examples include MPBH (4-(4-N maleimidophenyl)butyric acid hydrazide) and PDPH (4-(N-maleimidomethyl)cyclohexane-1-carboxyl-hydrazide (M2C2H and 3-2(2-pyridyldithio)proprionyl hydrazide). One may further cite ASIB (1-(p azidosalicylamido)-4-(iodoacetamido)butyrate), or the thiol reactive reagents described in Frisch et al. (Bioconjugate Chem. 7 (1996) 180-186).

In a preferred embodiment, the compound of the invention is of formula VII: 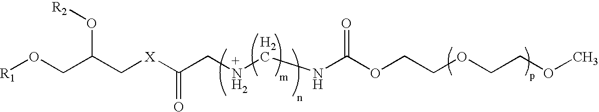
in which R1, R2, X, n and m are as mentioned above, p is a positive integer from 4 to 220, preferably from 22 to 110 and more preferably about 44.

According to an advantageous embodiment, the compound of the invention is of formula VIII: 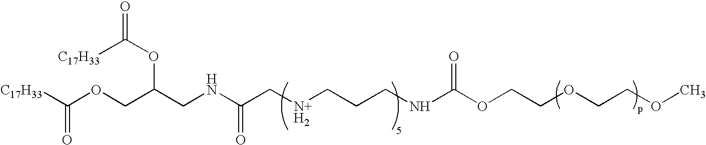
in which p is a positive integer from 4 to 220, preferably from 22 to 110 and more preferably is 44 (pcTG231).

The present invention further concerns complexes comprising (a) at least one compound of the present invention and (b) at least one substance of interest.

“Substance of interest” designates preferably a charged molecule without limitation of the number of charges. Preferably, said molecule is an anionic substance of interest, and more preferably it is selected from the group consisting of proteins and nucleic acid molecules. According to a preferred embodiment, said anionic substance of interest is a nucleic acid molecule.

The term “nucleic acid” or “nucleic acid molecule” as used in the scope of the present invention means a DNA or RNA or a fragment or combination thereof, which is single- or double-stranded, linear or circular, natural or synthetic, modified or not (see U.S. Pat. No. 5,525,711, U.S. Pat. No. 4,711,955, U.S. Pat. No. 5,792,608 or EP 302 175 for modification examples) without size limitation. It may, inter alia, be a genomic DNA, a cDNA, an mRNA, an antisense RNA, a ribozyme, or a DNA encoding such RNAs. The terms “polynucleotide”, “nucleic acid molecule” and “nucleic acids” are synonyms with regard to the present invention. The nucleic acid may be in the form of a linear or circular polynucleotide, and preferably in the form of a plasmid. The nucleic acid can also be an oligonucleotide which is to be delivered to the cell, e.g., for antisense or ribozyme functions. According to the invention, the nucleic acid is preferably a naked polynucleotide (Wolff et al., Science 247 (1990), 1465-1468) or is formulated with at least one compound such as a polypeptide, preferably a viral polypeptide, or a cationic lipid, or a cationic polymer, or combination thereof, which can participate in the uptake of the polynucleotide into the cells (see Ledley, Human Gene Therapy 6 (1995), 1129-1144 for a review) or a protic polar compound (examples are provided below in the present application or in EP 890362). Alternatively, nucleic acid further designate a viral vector (adenoviral vector, retroviral vector, poxviral vector, etc . . . ). The term <<viral vector>> as used in the present invention encompasses the vector genome, the viral particles (i.e. the viral capsid including the viral genome) as well as empty viral capsids.

“Plasmid” refers to an extrachromosomic circular DNA. The choice of the plasmids is very large. Plasmids can be purchased from a variety of manufacturers. Suitable plasmids include but are not limited to those derived from pBR322 (Gibco BRL), pUC (Gibco BRL), pBluescript (Stratagene), pREP4, pCEP4 (Invitrogene), pCI (Promega) and p Poly (Lathe et al., Gene 57 (1987), 193-201). It is also possible to engineer such a plasmid by molecular biology techniques (Sambrook et al., Laboratory Manual, Cold Spring Harbor Laboratory Press, Cold Spring Harbor (1989), N.Y.). A plasmid may also comprise a selection gene in order to select or identify the transfected cells (e.g. by complementation of a cell auxotrophy, antibiotic resistance), stabilizing elements (e.g. cer sequence; Summers and Sherrat, Cell 36 (1984), 1097-1103) or integrative elements (e.g. LTR viral sequences).

Preferably, said nucleic acid molecule includes at least one encoding gene sequence of interest (i.e. a transcriptional unit) that can be transcribed and translated to generate a polypeptide of interest and the elements enabling its expression (i.e. an expression cassette). If the nucleic acid contains this proper genetic information when it is placed in an environment suitable for gene expression, its transcriptional unit will thus express the encoded gene product. The level and cell specificity of expression will depend to a significant extent on the strength and origin of the associated promoter and the presence and activation of an associated enhancer element. Thus in a preferred embodiment, the transcriptional control element includes the promoter/enhancer sequences such as the CMV promoter/enhancer. However, those skilled in the art will recognize that a variety of other promoter and/or enhancer sequences are known which may be obtained from any viral, prokaryotic, e.g. bacterial, or eukaryotic organism, which are constitutive or regulable, which are suitable for expression in eukaryotic cells, and particularly in target cells or tissues. More precisely, this genetic information necessary for expression by a target cell or tissue comprises all the elements required for transcription of said gene sequence (if this gene sequence is DNA) into RNA, preferably into mRNA, and, if necessary, for translation of the mRNA into a polypeptide. Promoters suitable for use in various vertebrate systems are widely described in literature. Suitable promoters include but are not limited to the adenoviral E1a, MLP, PGK (Phospho Glycero Kinase; Adra et al. Gene 60 (1987) 65-74; Hitzman et al. Science 219 (1983) 620-625), RSV, MPSV, SV40, CMV or 7.5k, the vaccinia promoter, inducible promoters, MT (metallothioneine; Mc Ivor et al., Mol. Cell Biol. 7 (1987), 838-848), alpha-1 antitrypsin, CFTR, immunoglobulin, alpha-actin (Tabin et al., Mol. Cell Biol. 2 (1982), 426-436), SR (Takebe et al., Mol. Cell. Biol. 8 (1988), 466-472), early SV40 (Simian Virus), RSV (Rous Sarcoma Virus) LTR, TK-HSV-1, SM22 (WO 97/38974), Desmin (WO 96/26284) and early CMV (Cytomegalovirus; Boshart et al. Cell 41 (1985) 521), etc. Alternatively, one may use a synthetic promoter such as those described in Chakrabarti et al. (1997, Biotechniques 23, 1094-1097), Hammond et al. (1997, J. Virological Methods 66, 135-138) or Kumar and Boyle (1990, Virology 179, 151-158) as well as chimeric promoters between early and late poxviral promoters. Alternatively, promoters can be used which are active in tumor cells. Suitable examples include but are not limited to the promoters isolated from the gene encoding a protein selected from the group consisting of MUC-1 (overexpressed in breast and prostate cancers; Chen et al., J. Clin. Invest. 96 (1995), 2775-2782), CEA (Carcinoma Embryonic Antigen; overexpressed in colon cancers; Schrewe et al., Mol. Cell. Biol. 10 (1990), 2738-2748), tyrosinase (overexpressed in melanomas; Vile et al., Cancer Res. 53 (1993), 3860-3864), ErbB-2 (overexpressed in breast and pancreas cancers; Harris et al., Gene Therapy 1 (1994), 170-175) and alpha-foetoprotein (overexpressed in liver cancers; Kanai et al., Cancer Res. 57 (1997), 461-465) or combinations thereof. The early CMV promoter is preferred in the context of the invention.

The nucleic acid can also include intron sequences, targeting sequences, transport sequences, sequences involved in replication or integration. Said sequences have been reported in the literature and can readily be obtained by those skilled in the art. The nucleic acid can also be modified in order to be stabilized with specific components, for example spermine. It can also be substituted, for example by chemical modification, in order to facilitate its binding with specific polypeptides such as, for example the peptides of the present invention. According to the invention, the nucleic acid can be homologous or heterologous to the target cells into which it is introduced.

In a preferred embodiment, the nucleic acid contains at least one gene sequence of interest encoding a gene product which is a therapeutic molecule (i.e. a therapeutic gene). A “therapeutic molecule” is one which has a pharmacological or protective activity when administered, or expressed, appropriately to a patient, especially patient suffering from a disease or illness condition or who should be protected against this disease or condition. Such a pharmacological or protective activity is one which is expected to be related to a beneficial effect on the course or a symptom of said disease or said condition. When the skilled man selects in the course of applying the present invention a gene encoding a therapeutic molecule, he generally relates his choice to results previously obtained and can reasonably expect, without undue experiment other than practicing the invention as claimed, to obtain such pharmacological property. According to the invention, the sequence of interest can be homologous or heterologous to the target cells into which it is introduced. Advantageously said sequence of interest encodes all or part of a polypeptide, especially a therapeutic or prophylactic polypeptide giving a therapeutic or prophylactic effect. A polypeptide is understood to be any translational product of a polynucleotide regardless of size, and whether glycosylated or not, and includes peptides and proteins. Therapeutic polypeptides include as a primary example those polypeptides that can compensate for defective or deficient proteins in an animal or human organism, or those that act through toxic effects to limit or remove harmful cells from the body. They can also be immunity conferring polypeptides which act as an endogenous antigen to provoke a humoral or cellular response, or both.

The following encoding gene sequences are of particular interest. For example genes coding for a cytokine (α,β or γ-interferon, interleukine (IL), in particular IL-2, IL-6, IL-10 or IL-12, a tumor necrosis factor (TNF), a colony stimulating factor (such as GM-CSF, C-CSF, M-CSF), an immunostimulatory polypeptide (such as B7.1, B7.2, CD40, CD4, CD8, ICAM and the like), a cell or nuclear receptor, a receptor ligand (such as fas ligand), a coagulation factor (such as FVIII, FIX), a growth factor (such as Transforming Growth Factor TGF, Fibroblast Growth Factor FGF and the like), an enzyme (such as urease, renin, thrombin, metalloproteinase, nitric oxide synthase NOS, SOD, catalase), an enzyme inhibitor (such as α1-antitrypsine, antithrombine III, viral protease inhibitor, plasminogen activator inhibitor PAI-1), the CFTR protein, insulin, dystrophin, a MHC antigen (Major Histocompatibility Complex) of class I or II or a polypeptide that can modulate/regulate the expression of one or more cellular genes, a polypeptide capable of inhibiting a bacterial, parasitic or viral infection or its development (such as antigenic polypeptides, antigenic epitopes, transdominant variants inhibiting the action of a native protein by competition), an apoptosis inducer or inhibitor (such as Bax, Bcl2, BclX), a cytostatic agent (such as p21, p16, Rb), an apolipoprotein (such as ApoAI, ApoAIV, ApoE), an inhibitor of angiogenesis (such as angiostatin, endostatin), an angiogenic polypeptide (such as family of Vascular Endothelial Growth Factors VEGF, FGF family, CCN family including CTGF, Cyr61 and Nov), an oxygen radical scavenger, a polypeptide having an anti-tumor effect, an antibody, a toxin, an immunotoxin and a marker (such as beta-galactosidase, luciferase) or any other gene of interest that is recognized in the art as being useful for the treatment or prevention of a clinical condition. In view of treating a hereditary dysfunction, one may use a functional allele of a defective gene, for example a gene encoding factor VIII or IX in the context of haemophilia A or B, dystrophin (or minidystrophin) in the context of myopathies, insulin in the context of diabetes, CFTR (Cystic Fibrosis Transmembrane Conductance Regulator) in the context of cystic fibrosis. Suitable anti-tumor genes include but are not limited to those encoding an antisense RNA, a ribozyme, a cytotoxic product such as thymidine kinase of herpes-1 simplex virus (TK-HSV-1), ricin, a bacterial toxin, the expression product of yeast genes FCY1 and/or FUR1 having UPRTase (Uracile Phosphoribosyltransferase) and CDase (Cytosine Deaminase) activity respectively, an antibody, a polypeptide inhibiting cellular division or transduction signals, a tumor suppressor gene (p53, Rb, p73), a polypeptide activating host immune system, a tumor-associated antigen (MUC-1, BRCA-1, an HPV early or late antigen (E6, E7, L1, L2), optionally in combination with a cytokine gene. The polynucleotide can also encode an antibody. In this regard, the term “antibody” encompasses whole immunoglobulins of any class, chimeric antibodies and hybrid antibodies with dual or multiple antigen or epitope specificities, and fragments, such as F(ab)′₂, Fab′, Fab including hybrid fragments and anti-idiotypes (U.S. Pat. No. 4,699,880). Advantageously said nucleic acid encodes all or part of a polypeptide which is an immunity conferring polypeptide and acts as endogenous immunogen to provoke a humoral or cellular response, or both, against infectious agents, including intracellular viruses, or against tumor cells. An “immunity-conferring polypeptide” means that said polypeptide when it is produced in the transfected cells will participate in an immune response in the treated patient. More specifically, said polypeptide produced in or taken up by macropinocyte cells such as APCs will be processed and the resulting fragments will be presented on the surface of these cells by MHC class I and/or II molecules in order to elicit a specific immune response.

The nucleic acid may comprise one or more gene(s) of interest. In this regard, the combination of genes encoding a suicide gene product and a cytokine gene (e.g. α, β or γ interferons, interleukins, preferably selected among IL-2, IL-4, IL-6, IL-10 or IL-12, TNF factors, GM-CSF, C-CSF, M-CSF and the like), an immunostimulatory gene (e.g. B7.1, B7.2, ICAM) or a chimiokine gene (e.g. MW, RANTES, MCP 1) is advantageous. The different gene expression may be controlled by a unique promoter (polycistronic cassette) or by independent promoters. Moreover, they may be inserted in a unique site or in various sites along the nucleic acid either in the same or opposite directions.

The encoding gene sequence of interest may be isolated from any organism or cell by conventional techniques of molecular biology (PCR, cloning with appropriate probes, chemical synthesis) and if needed its sequence may be modified by mutagenesis, PCR or any other protocol.

Alternatively, the “substance of interest” is a peptide (polypeptide, protein and peptide are synonyms) including variant or modified peptides, peptide-like molecules, antibodies or fragments thereof, chimeric antibody. Within the context of the present invention, preferred proteins are those able to inhibit restenosis, hypertension, to improve heart contracting activity or heart cell survival (e.g. angiogenic factors, cellular receptors or channels involved in ion homeostasis).

The term “complex” refers to molecular assemblages of at least one compound of the present invention and at least one charged, preferably anionic, substance which are bound to one another preferably in a reversible manner, for example by ionic interactions, by forming disulfide or hydrogen bonds, by hydrophobic interactions or covalent bonds. Preferably, a compound of the invention is capable of interacting and binding to an anionic substance of interest at least by the intermediate of ionic interactions. Such a complex may contain further elements which are described in the followings.

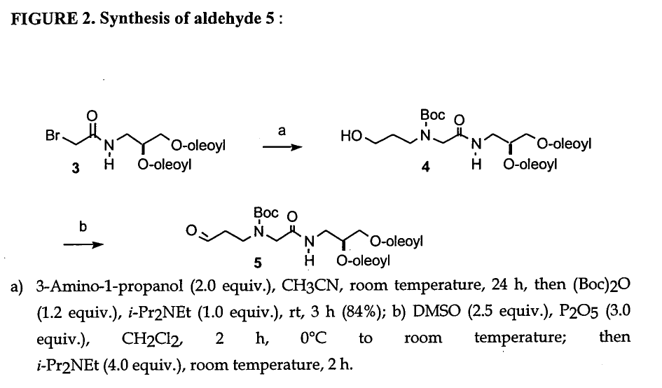

As shown in the Experimental section, the complexes of the present invention present the advantageous property to reduce, and in preferred case to eliminate, non-specific transfer of substances of interest into cells (see for example FIG. 2).

According to a particular embodiment, the complex of the invention further comprises:
(c) at least one substituting moiety; and/or
(d) at least one targeting component; and/or
(e) at least one peptide which is capable of causing membrane disruption; and/or
(f) at least one cationic compound selected from the group consisting of cationic lipids and cationic polymers; and/or
(g) at least one colipid.

The substituting moiety (c) can be present on any of the elements comprised in the complex of the invention. According to a preferred embodiment, at least one compound of the present invention comprised into said complex is substituted with at least one substituting moiety. By extension, the present invention therefore further concerns such a substituted compound of the present invention. Said substitution can actually consist in the addition of at least one labelling molecule (for example, see molecules disclosed in U.S. Pat. No. 4,711,955) enabling, for example, visualisation of the distribution of the compounds, or of complexes incorporating them, after in vitro or in vivo administration; a tissue and/or cell targeting molecule (i.e. a ligand) or an anchoring molecule. Such substituting elements, which have been widely described in scientific publications, allow targeting of a specific cell and/or tissue type, facilitating penetration into the cell, lysis of endosomes or even intracellular transport towards the nucleus. These elements may be composed of all or part of sugars, glycol, peptides (e.g. GRP, Gastrin Releasing Peptide), oligonucleotides, lipids, hormones, vitamins, antigens, antibodies (or fragments thereof), specific membrane receptor ligands, ligands capable of reaction with an anti-ligand, fusogen peptides, nuclear localization peptides, or a combination of said compounds, e.g. galactosyl residues to target the asialoglycoprotein receptor on the surface of hepatocytes, the INF-7 fusogen peptide derivated from the HA-2 subunit of the influenza virus hemagglutinin (Plank et al. 1994, J. Biol. Chem. 269, 12918-12924) for membrane fusion, or a nuclear signal sequence derived from the T-antigen of the SV40 virus (Lanford and Butel, 1984, Cell 37, 801-813) or from the EBNA-1 protein of the Epstein Barr virus (Ambinder et al., 1991, J. Virol. 65, 1466-1478).

In one specific embodiment, the substitution occurs at least on one of the secondary or primary nitrogen atoms of the polar headgroup spacer of the compound according to the invention. Alternatively, substitution can occur on non-reactive groups, such as the carbon atoms in CH or CH₂. In preferred embodiment, said substitution is performed at the level of the hydrophilic polymer comprised in the disclosed compounds, and more preferably at one extremity of said polymer.

Incorporation of the substituting moiety can be performed during the synthesis of the one of the elements forming the complex of the invention, and more specifically synthesis of the claimed compounds, using methods familiar to skilled person (e.g. use of reactive groups, . . . ). Alternatively, it can also be performed on the neosynthesized compounds or on the neoformed complexes of the invention.

According to special embodiment, substituting moiety can be coupled to the compound of the invention, at the level of either the hydrophilic, hydrophobic or polar region, or combination hereof, as above described, by covalently or non-covalently links, including or not homobifunctional or heterobifunctional cross-linking reagent.

In a preferred embodiment of the invention, the substituting moiety is a ligand moiety able to recognise and to bind to cell and/or tissue. In a particular embodiment of the invention, the compound of the invention is substituted with a ligand moiety able to recognise and to bind to cell and/or tissue. The term “ligand moiety able to recognise and to bind to cell and/or tissue” refers to a ligand moiety which is able to recognise and to bind specifically with a cell membrane receptor (i.e. anti-ligand). Said cell membrane surface receptor is a molecule or structure which can bind said ligand with high affinity and preferably with high specificity. Said cell membrane surface receptor is preferably specific for a particular cell or tissue, i.e. it is found predominantly in one type of cells and/or tissues rather than in another type of cells and/or tissues (e.g. galactosyl residues to target the asialoglycoprotein receptor on the surface of hepatocytes). The cell membrane surface receptor facilitates cell and/or tissue targeting of any molecules coupled to said ligand (i.e. the compound or complex of the invention).

A large number of couple ligand moieties/anti-ligands that may be used in the context of the present invention are widely described in the literature. Such a ligand moiety is capable of conferring to the compound or to the complex of the invention, the ability to bind to a given anti-ligand molecule or a class of anti-ligand molecules localized at the surface of at least one target cell and/or target tissue. Suitable anti-ligand molecules include without limitation polypeptides selected from the group consisting of cell-specific markers, tissue-specific markers, cellular receptors, viral antigens, antigenic epitopes and tumor-associated markers. Anti-ligand molecules may moreover consist of or comprise one or more sugar, lipid, glycolipid or antibody molecules. According to the invention, a ligand moiety may be for example a lipid, a glycolipid, a hormone, a sugar, a polymer (e.g. PEG, polylysine, PEI), an oligonucleotide, a vitamin, an antigen, all or part of a lectin, all or part of a polypeptide such as for example JTS1 (WO 94/40958), an antibody or a fragment thereof, or a combination thereof.

According to one embodiment, the ligand moiety used in the present invention is a peptide or polypeptide having a minimal length of 7 amino acids. It is either a native polypeptide or a polypeptide derived from a native polypeptide. “Derived polypeptide” means that said polypeptide is containing (a) one or more modifications with respect to the sequence of the native polypeptide (e.g. addition, deletion and/or substitution of one or more residues), (b) amino acid analogs, including not naturally occurring amino acids or (c) substituted linkages or (d) other modifications known in the art, or combination of (a) to (d) cases. The polypeptides serving as ligand moiety encompass variant and chimeric polypeptides obtained by fusing sequences of various origins, such as for example a humanized antibody which combines the variable region of a mouse antibody and the constant region of a human immunoglobulin. In addition, such polypeptides may have a linear or cyclized structure (e.g. by flanking at both extremities a polypeptide ligand by cysteine residues). Additionally, the polypeptide in use as ligand moiety may include modifications of its original structure by way of substitution or addition of chemical moieties (e.g. glycosylation, alkylation, acetylation, amidation, phosphorylation, addition of sulfhydryl groups and the like). The invention further contemplates modifications that render the ligand moiety detectable. For this purpose, modifications with a detectable moiety can be envisaged (i.e. a scintigraphic, radioactive, or fluorescent moiety, or a dye label and the like). Suitable radioactive labels include but are not limited to Tc^{99m}, I¹²³ and In¹¹¹. Such detectable labels may be attached to the ligand moiety by any conventional techniques and may be used for diagnostic purposes (e.g. imaging of tumoral cells).

In one special embodiment, the anti-ligand molecule is an antigen (e.g. a target cell-specific antigen, a disease-specific antigen, an antigen specifically expressed on the surface of engineered target cells) and the ligand moiety is an antibody, a fragment or a minimal recognition unit thereof (i.e. a fragment still presenting an antigenic specificity) such as those described in detail in immunology manuals (see for example Immunology, third edition 1993, Roitt, Brostoff and Male, ed Gambli, Mosby). The ligand moiety may be a monoclonal antibody. Monoclonal antibodies which will bind to many of these antigens are already known but in any case, with today's techniques in relation to monoclonal antibody technology, antibodies may be prepared to most antigens. The ligand moiety may be a part of an antibody (for example a Fab fragment) or a synthetic antibody fragment (for example, ScFv).

Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: Techniques and Applications”, J. G. R. Hurrell (CRC Press, 1982). Suitably prepared non-human antibodies may be “humanized” in known ways, for example by inserting the CDR regions of mouse antibodies into the framework of human antibodies. Additionally, as the variable heavy (VH) and variable light (VL) domains of the antibody are involved in antigen recognition, variable domains of rodent origin may be fused to constant domains of human origin such that the resultant antibody retains the antigenic specificity of the rodent parental antibody (Morrison et al (1984) Proc. Natl. Acad. Sci. USA 81, 6851-6855).

Antigenic specificity of antibodies is conferred by variable domains including Fab-like molecules (Better et al (1988) Science 240, 1041); Fv molecules (Skerra et al (1988) Science 240, 1038); ScFv molecules where the VH and VL partner domains are linked via a flexible oligopeptide (Bird et al (1988) Science 242, 423; Huston et al (1988) Proc. Natl. Acad. Sci. USA 85, 5879) and dAbs comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

According to an advantageous embodiment, the ligand moiety is selected among antibody fragments, rather than whole antibodies. Effective functions of whole antibodies, such as complement binding, are removed. ScFv and dAb antibody fragments may be expressed as a fusion with one or more other polypeptides. Minimal recognition units may be derived from the sequence of one or more of the complementary-determining regions (CDR) of the Fv fragment. Whole antibodies, and F(ab′)2 fragments are “bivalent”. By “bivalent” we mean that said antibodies and F(ab′) 2 fragments have two antigen binding sites. In contrast, Fab, Fv, ScFv, dAb fragments and minimal recognition units are monovalent, having only one antigen binding sites.

In another embodiment, the ligand moiety allows to target a virally infected cell and is capable of recognizing and binding to a viral component (e.g. envelope glycoprotein) or capable of interfering with the virus biology (e.g. entry or replication). For example, the targeting of an HIV (Human Immunodeficiency Virus) infected cell can be performed with a ligand moiety specific for an epitope of the HIV envelope, such as a ligand moiety derived from the 2F5 antibody (Buchacher et al., 1992, Vaccines 92, 191-195) recognizing a highly conserved epitope of the transmembrane glycoprotein gp41 or with a ligand moiety interfering with HIV attachment to its cellular receptor CD4 (e.g. the extracellular domain of the CD4 molecule).

In another embodiment, the ligand moiety allows to target a tumor cell and is capable of recognizing and binding to a molecule related to the tumor status, such as a tumor-specific antigen, a cellular protein differentially or over-expressed in tumor cells or a gene product of a cancer-associated virus.

Examples of tumor-specific antigens include but are not limited to MUC-1 related to breast cancer (Hareuveni et al., 1990, Eur. J. Biochem 189, 475-486), the products encoded by the mutated BRCA1 and BRCA2 genes related to breast and ovarian cancers (Miki et al., 1994, Science 226, 66-71; Futreal et al., 1994, Science 226, 120-122; Wooster et al., 1995, Nature 378, 789-792), APC related to colon cancer (Polakis, 1995, Curr. Opin. Genet. Dev. 5, 66-71), prostate specific antigen (PSA) related to prostate cancer, (Stamey et al., 1987, New England J. Med. 317, 909), carcinoma embryonic antigen (CEA) related to colon cancers (Schrewe et al., 1990, Mol. Cell. Biol. 10, 2738-2748), tyrosinase related to melanomas (Vile et al., 1993, Cancer Res. 53, 3860-3864), receptor for melanocyte-stimulating hormone (MSH) which is expressed in high number in melanoma cells, ErbB-2 related to breast and pancreas cancers (Harris et al., 1994, Gene Therapy 1, 170-175), and alpha-foetoprotein related to liver cancers (Kanai et al., 1997, Cancer Res. 57, 461-465).

A special ligand moiety in use in the present invention is a fragment of an antibody capable of recognizing and binding to the MUC-1 antigen and thus targeting the MUC-1 positive tumor cells. A more preferred ligand moiety is the scFv fragment of the SM3 monoclonal antibody which recognizes the tandem repeat region of the MUC-1 antigen (Burshell et al., 1987, Cancer Res. 47, 5476-5482; Girling et al., 1989, Int J. Cancer 43, 1072-1076; Dokurno et al., 1998, J. Mol. Biol. 284, 713-728).

Examples of cellular proteins differentially or overexpressed in tumor cells include but are not limited to the receptor for interleukin 2 (IL-2) overexpressed in some lymphoid tumors, GRP (Gastrin Release Peptide) overexpressed in lung carcinoma cells, pancreas, prostate and stomach tumors (Michael et al., 1995, Gene Therapy 2, 660-668), TNF (Tumor Necrosis Factor) receptor, epidermal growth factor receptors, Fas receptor, CD40 receptor, CD30 receptor, CD27 receptor, OX-40, αv integrins (Brooks et al., 1994, Science 264, 569) and receptors for certain angiogenic growth factors (Hanahan, 1997, Science 277, 48). Based on these indications, it is within the scope of those skilled in the art to define an appropriate ligand moiety capable of recognizing and binding to such proteins. To illustrate, IL-2 is a suitable ligand moiety to bind to IL-2 receptor.

Suitable gene products of cancer-associated viruses include but are not limited to human papilloma virus (HPV) E6 and E7 early polypeptides as well as L1 and L2 late polypeptides (EP 0 462 187, U.S. Pat. No. 5,744,133 and WO98/04705) that are expressed in cervical cancer and EBNA-1 antigen of Epstein-Barr virus (EBV) associated with Burkitt's lymphomas (Evans et al., 1997, Gene Therapy 4, 264-267).

In still another embodiment, the ligand moiety allows to target tissue-specific molecules. For example, ligand moieties suitable for targeting liver cells include but are not limited to those derived from ApoB (apolipoprotein) capable of binding to the LDL receptor, alpha-2-macroglobulin capable of binding to the LPR receptor, alpha-1 acid glycoprotein capable of binding to the asialoglycoprotein receptor and transferrin capable of binding to the transferrin receptor. A ligand moiety for targeting activated endothelial cells may be derived from the sialyl-Lewis-X antigen (capable of binding to ELAM-1), from VLA-4 (capable of binding to the VCAM-1 receptor) or from LFA-1 (capable of binding to the ICAM-1 receptor). A ligand moiety derived from CD34 is useful to target hematopoïetic progenitor cells through binding to the CD34 receptor. A ligand moiety derived from ICAM-1 is more intended to target lymphocytes through binding to the LFA-1 receptor. Finally, the targeting of T-helper cells may use a ligand moiety derived from HIV gp-120 or a class II MHC antigen capable of binding to the CD4 receptor.

In a preferred embodiment of the invention, the compound of the invention is substituted with a ligand able to recognise and to bind specifically with a cell membrane receptor wherein said membrane receptor is an adrenoceptor.

Adrenoceptors are cell membrane receptors for neurotransmitters and hormones belonging to the catecholamine family. Actually, adrenoceptors can be divided into two subtypes, i.e. the α and β adrenoceptors.

α adrenoceptors (including α1 and α2 adrenoceptor subtypes) are located on cells or tissue of the central and peripheral nervous system. For example, α1 adrenoceptors are located on cells or tissues of vascular and non-vascular smooth muscle, heart and liver. α2 adrenoceptors are located on pre- and post-synaptic neurones.

β adrenoceptors includes β1, β2, β3 and β4 subtypes. β1 adrenoceptors are located on cells or tissue of heart and adipose tissues. β2 adrenoceptors are located on cells or tissue of vascular, uterine and airway smooth muscles. β3 adrenoceptors are located on cells of adipose tissue. β4 adrenoceptors are located on cells or tissue of heart.

Ligands able to recognise and bind specifically with adrenoceptors are disclosed in litterature however their known applications are limited to pharmaceutical uses as adrenoceptor agonists or adrenoceptor antagonists; their application as ligand for targeting transfer of substances of interest towards or into cells, or tissue, expressing them has neither been disclosed nor suggested. Applicants have now demonstrated that this latter application is perfectly workable, especially in the frame of nucleic acid transfer into cells expressing adrenoceptors, for example cardiomyocyte cells. Thus the present invention further concerns the use of ligands able to recognise and bind specifically with adrenoceptors (i.e. α adrenoceptors or β adrenoceptors) as ligand for targeting transfer of substances of interest towards or into cells, or tissue, expressing them.

Examples of ligands allowing to target cells or tissues expressing adrenoceptor on their surface are provided in the following Table I:

| Adrenoceptors | ligands |
|---|---|
| α1 | Oxymetazoline |
| | A61603 |
| | KMD3213 |
| | CEC |
| | BMY7378 |
| | SKF105854 |
| | Corynanthine |
| | Prazosin |
| | Cirazoline |
| | M-6434 |
| | Methoxamine |
| | Phenylephrine |
| | RS17053 |
| | WB4101 |
| | (S)-(+)-niguldipine |
| | 5-methylurapidil |
| | SNAP5089 |
| | Rec152739 |
| | SB216469 |
| | Ro700004 |
| α2 | Oxymethazoline |
| | Guanfacine |
| | UK14,304 |
| | Clonidine |
| | BRL44408 |
| | BRL48962 |
| | ARC239 |
| | imixolan |
| | Rauwolscine |
| | MK912 |
| | RS79948 |
| | Yohimbine |
| | RS15385 |
| | RX821002 |
| | SKF86466 |
| | prazosin |
| β1 | Xamoterol |
| | Denopamine |
| | Betaxolol |
| | Bisoprolol |
| | Atenolol |
| | Practolol |
| | CGP20712A |
| β2 | Clenbuterol |
| | ICI 118551 |
| | Procaterol |
| | Salbutamol |
| | Salmeterol |
| | Formoterol |
| | Terbutaline |
| | Fenoterol |
| β3 | BRL37344 |
| | SR59230A |
| | ZD7114 |
| | CGP12177 |
| | CL316243 |
| β4 | Bupranolol |
| | CGP20712A |

Derivatives from said ligands can be used according to the present invention as far as these derivatives are still able to recognize and bind with the adrenoceptors as does the original ligand. This property can easily be analyzed as done in the Experimental Section of the present application, for example by competitive experiments. The chemical structure of said compounds is easily available to the skilled person and their synthesis is possible. Ligands are commercially available. See for example the Sigma (St Louis, USA) catalogue (http://www.sigma-aldrich.com/):
5-methylurapidil, Sigma reference: U101; full name: 5-Methyl-6[[3-[4-(2-methoxyphenyl)-1-piperazinyl]propyl]amino]-1,3-dimethyluracil
CL 316243, Sigma reference: C5976; full name: 5-[(2R)-2-([(2R)-2-(3-Chlorophenyl)-2-hydroxyethyl]amino)propyl]-1,3-benzodioxole-2,2-dicarboxylate
Oxymetazoline, Sigma reference: O2378; full name: 2-(3-Hydroxy-2,6-dimethyl-4-t-butylbenzyl)-2-imidazoline
BMY 7378, Sigma reference: B134; full name: 8-(2-[4-(2-Methoxyphenyl)-1-piperazinyl]ethyl)-8-azaspiro(4.5)decane-7,9-dion
BRL 37344A, Sigma reference: B169; full name: (R*,R*)-(4-[2-([2-(3-Chlorophenyl)-2-hydroxyethyl]amino)propyl]phenoxy)acetic acid
CGP-20712A, Sigma reference: C231; full name: (±)-2-Hydroxy-5-[2-[[2-hydroxy-3-[4-[1-methyl-4-(trifluoromethyl)-1H-imidazol-2-yl]phenoxy]propyl]amino]ethoxy]-benzamide methanesulfonate
Fenoterol, Sigma reference: F1016; full name: Hydrobromide 2-(3,5-Dihydroxyphenyl)-2-hydroxy-2′-(4-hydroxyphenyl)-1′-methyldiethylamine
Clenbuterol, Sigma reference: C5423; full name: 4-Amino-alpha-(t-butylaminomethyl)-3,5-dichlorobenzyl alcohol
Clonidine, Sigma reference: C7897; full name: 2-(2,6-Dichloroaniline)-2-imidazoline
ICI-118,551, Sigma reference: I127; full name: (±)-1-[2,3-(Dihydro-7-methyl-1H-inden-4-yl)oxy]-3-[(1-methylethyl)amino]-2-butanol
Prazosin, Sigma reference: P7791; full name: 1-(4-Amino-6,7-dimethoxy-2-quinazolinyl)-4-(2-furanylcarbonyl)piperazine
Salbutamol, Sigma reference: S8260; full name: alpha-([t-Butylamino]methyl)-4-hydroxy-m-xylene-alpha,alpha′-diol
UK 14,304, Sigma reference: U 104; full name: Brimonidine or 5-Bromo-N-(4,5-dihydro-1H-imidazol-2-yl)-6-quinoxalinamine

In preferred embodiments, the ligand able to recognize and to bind to adrenoceptor is atenolol or one of its derivatives (see for example Sigma reference: A7655). Atenolol complete name is 4-(2′-Hydroxy-3′-[isopropylamino]propoxy)phenylacetamide or molecular formula: C14H22N2O3. Its molecular weight is 266.3 (Allibardi et al., Pharmacol. Res., 39, 43 (1999); Smith and Teitler, Cardiovasc. Drugs Ther., 13, 123-126 (1999). More specifically, atenolol derivative can be the part of said molecule which is responsible of the adrenoceptor recognition and binding. For example, said atenolol derivative ligand can be of the following formula IX:

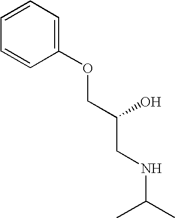

In a preferred embodiment, the ligand of formula IX is coupled to the compound of the present invention via a carbon atom of the benzene ring, and more preferably via the carbon atom in meta.

According to preferred embodiment, the invention concerns a complex as previously defined which comprises at least one substituted compound of the invention as disclosed above, especially those comprising ligand deriving from atenolol.

Alternatively, the substituting ligand coupled to the compound and/or complex of the invention can be all or part of a specific antibody which is able to bind with adrenoceptor. Such antibodies are well known to the one skilled in the art and are commercially available, see for example antibodies PA1-047, PA1-048 or PA1-049 from Affinity BioReagents, Inc. (Golden, USA). Additionally, such specific antibodies can be produced according to techniques widely used in the art (see for example, Antibodies—A Laboratory Manual, Harlow and Lane, eds., Cold Spring Harbor Laboratory, New York (1988).

By “target cells”, we refer to the cells that the complex or the compound of the invention can selectively target. Depending on the nature of the ligand moiety and/or of the anti-ligand molecule, “target cells” may designate a unique type of cell or a group of different types of cells (for example tissue) having as a common feature on their surface an anti-ligand molecule(s) recognized by ligand moiety(s) present in the complex of the invention. For the purpose of the invention, a target cell is any mammalian cell (preferably human cell) which can be targeted with a complex according to the present invention having a suitable ligand moiety. The term “to target” refers to addressing a certain type of cells or a group of types of cells (or tissues) for substance of interest transfer in favor of the remaining part of the totality of cells being contacted with the complex of the present invention. The target cell may be a primary cell, a transformed cell or a tumor cell. Suitable target cells include but are not limited to hematopoïetic cells (totipotent, stem cells, leukocytes, lymphocytes, monocytes, macrophages, APC, dendritic cells, non-human cells and the like), muscle cells (satellite, myocytes, myoblasts, skeletal or smooth muscle cells, heart cells), pulmonary cells, tracheal cells, hepatic cells, epithelial cells, endothelial cells or fibroblasts.

The substituting moiety can further be a ligand capable of nuclear targeting. Such a ligand is referring to a particular ligand which is capable of binding to a nuclear receptor (nuclear anti-ligand). Said nuclear receptor is a molecule or structure localized in or/and on the nuclear membrane which can bind to said ligand, thereby facilitating intracellular transport of the complex or compound of the present invention towards the nucleus and its internalization into the nucleus. Examples of such a ligand involved in nuclear targeting are the nuclear signal sequences derived from the T-antigen of the SV40 virus (Lanford and Butel, 1984, Cell 37, 801-813) and from the EBNA-1 protein of the Epstein Barr virus (Ambinder et al., 1991, J. Virol. 65, 1466-1478).

In a preferred alternative embodiment, the complex of the invention comprises a targeting component (d). Such a component (d) is formed of at least two distinct parts: the first one which is responsible of the targeting of the complex and the second which is a carrier permitting incorporation of said targeting component into the complex. The “targeting part” can be all or part of the above mentionned substituting moieties, and preferably one of the above described ligands. Preferably, said targeting part is a ligand able to recognise and to bind to cell and/or tissue, such as for example those above disclosed. The “carrier part” can be any carrier which is able to be incorporated into the complex. More specifically, said carrier might be a charged, a zwitterionic or a non charged compound. It might comprise alkyl or alkenyl chains; it might for example comprise hydrophilic element such as for example those above described; it might further comprise any spacer molecule

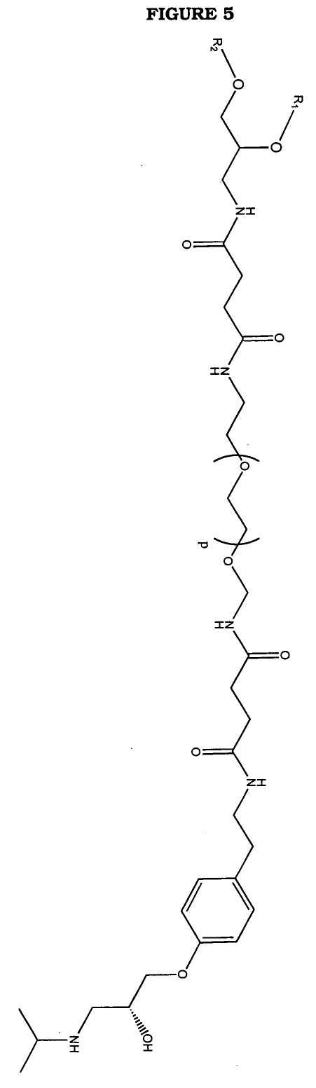

In a preferred embodiment, the complex of the present invention comprises a targeting component (d) wherein the targeting part is an atenolol derivative ligand. More preferably, the complex of the present invention comprises a targeting component (d) of formula X (i.e. pcTG238 ) as shown in FIG. 5 in which p is a positive integer from 4 to 220, preferably from 22 to 110 and more preferably about 44.

In one special embodiment, the complex of the invention may comprise (e) at least one peptide capable of causing membrane disruption. Examples of such peptides are JTS-1, JTS-1-K13, GALA, KALA, ppTG1 and related peptides (see Mahato et al., 1999, Current Opinion in Mol. Therapeutics 1, 226-243; WO 96/40958; WO 98/50078; Gottschalk et al., 1996, Gene Therapy, 3, 448-457; Haensler & Szoka, 1993, Bioconjugate Chem., 4, 372-379; Wyman et al., 1997, Biochemistry, 36, 3008-3017, patent application EP 01 44 0049.3).

In another embodiment, the complex of the invention may further comprise (f) at least one cationic compound selected from the group consisting of cationic lipids and cationic polymers. Cationic compounds are widely described in the scientific literature (see for example the references related to non-viral delivery systems mentioned above, or WO 97/29118, WO 98/08489, WO 98/17693 and those previously cited in the present specification). Cationic lipids or mixtures of cationic lipids which may be used in the present invention include cationic lipids selected from the group consisting of Lipofectin™, DOTMA: N-[1-(2,3-dioleyloxyl)propyl]-N,N,N-trimethylammonium (Felgner, PNAS 84 (1987), 7413-7417), DOGS: dioctadecylamidoglycylspermine or Transfectam™ (Behr, PNAS 86 (1989), 6982-6986), DMRIE: 1,2-dimiristyloxypropyl-3-dimethyl-hydroxyethylammonium and DORIE: 1,2-diooleyloxypropyl-3-dimethyl-hydroxyethylammnoium (Felgner, Methods 5 (1993), 67-75), DC-CHOL: 3 [N-(N′,N′-dimethylaminoethane)-carbamoyl]cholesterol (Gao, BBRC 179 (1991), 280-285), DOTAP (McLachlan, Gene Therapy 2 (1995), 674-622), Lipofectamine™, spermine or spermidine-cholesterol, Lipofectace™ (for a review see for example Legendre, Medecine/Science 12 (1996), 1334-1341 or Gao, Gene Therapy 2 (1995), 710-722), cationic lipid as disclosed in patent applications WO 98/34910, WO 98/14439, WO 97/19675, WO 97/37966 and their isomers. Nevertheless, this list is not exhaustive and other cationic lipids well known in the art can be used in connection with the present invention as well. Cationic polymers or mixtures of cationic polymers which may be used in the present invention include cationic polymers selected from the group consisting of chitosan, poly(aminoacids) such as polylysine (U.S. Pat. No. 5,595,897 and FR 2 719 316); polyquaternary compounds; protamine; polyimines; polyethylene imine or polypropylene imine (WO 96/02655); polyvinylamines; polycationic polymer derivatized with DEAE, such as pullulans, celluloses; polyvinylpyridine; polymethacrylates; polyacrylates; polyoxethanes; polythiodiethylaminomethylethylene (P(TDAE)); polyhistidine; polyornithine; poly-p-aminostyrene; polyoxethanes; co-polymethacrylates (eg copolymer of HPMA; N-(2-hydroxypropyl)-methacrylamide); the compound disclosed in U.S. Pat. No. 3,910,862, polyvinylpyrrolid complexes of DEAE with methacrylate, dextran, acrylamide, polyimines, albumin, onedimethylaminomethylmethacrylates and polyvinylpyrrolidone-methylacrylaminopropyltrimethyl ammonium chlorides; polyamidoamines; telomeric compounds. Nevertheless, this list is not exhaustive and other cationic polymers well known in the art can be used in connection with the present invention as well.

Colipids (g) may be optionally included in the complex of the invention in order to facilitate entry of the nucleic acid into the cell. According to the invention, colipids are selected from the group consisting of positively or negatively charged, neutral or zwitterionic lipids. These colipids are, for example, selected from the group consisting of phosphatidylethanolamine (PE), phosphatidylcholine, phosphocholine, dioleylphosphatidylethanolamine (DOPE), sphingomyelin, ceramide or cerebroside and one of their derivatives.

The various elements of the complex (i.e. ligand, anionic or cationic compounds, colipid, compound of the invention, anionic substance of interest) may be modified or substituted by chemical or natural processes widely used by the skilled man in order to obtain compounds modified or substituted such as those disclosed above, enabling, for example, visualization of the distribution of the polypeptide expressed by the nucleic acid, of the nucleic acid, or of the complex of the invention, after in vitro or in vivo administration of the complex.

In a specific embodiment of the invention, the size of the complex according to the invention is small (i.e. its diameter is less than 2 μm, preferably less than 500 nm and, most preferably, it ranges between 20 and 100 nm). The size of the complex may be selected for optimal use in particular applications. Measurements of the complex size can be achieved by a number of techniques including, but not limited to, dynamic laser light scattering (photon correlation spectroscopy, PCS), as well as other techniques known to those skilled in the art (see, Washington, Particle Size Analysis in Pharmaceutics and other Industries, Ellis Horwood, New York, 1992, 135-169). Sizing procedure may also be applied on complexes in order to select specific complex sizes. Methods which can be used in this sizing step include, but are not limited to, extrusion, sonication and microfluidization, size exclusion chromatography, field flow fractionation, electrophoresis and ultracentrifugation.

The ratios of cationic component or of compound of the present invention to colipid (on a mole to mole basis), when the two compounds are co-existing in the complex, can range from 1:0 to 1:10. In preferred embodiments, the ratio ranges from 1:0.5 to 1:4, advantageously said ratio is about 1:2.

The ratios of compound of the present invention to cationic component (e.g. cationic lipid) (on a mole to mole basis), when the two compounds are co-existing in the complex, can range from 1:0 to 1:0.005. In preferred embodiments, the ratio ranges from 1:0.5 to 1:0.05. In most preferred embodiment the ratio is 100% of compound of the present invention.

In case where the complex comprises a cationic component (e.g. cationic lipid, for example pcTG90) and a compound of the present invention coupled with a ligand the ratios of said compounds can range from 1:0.005 to 1:0.5, preferably from 1:0.01 to 1:0.2.

In case where the complex comprises a compound of the present invention (for example pcTG231) and a targeting component (for example pcTG238), the ratios of said compounds can range from 1:0.005 to 1:0.5, preferably from 1:0.01 to 1:0.2. Advantageously, said ratio ranges from 1:0.06 to 1:0.18.

In case where the complex comprises a cationic component (for example pcTG90) and a targeting component (for example pcTG238), the ratios of said compounds can range from 1:0.005 to 1:0.5, preferably from 1:0.01 to 1:0.2. Advantageously, said ratio ranges from 1:0.06 to 1:0.18.

The complexes of the invention may also be characterized by their theoretical charge ratio (+/−), which is the ratio of the positive charges provided by at least the positively charged compound to the negative charges provided by the anionic substance in the complex, assuming that all potentially cationic groups are in fact in the cationic state and all potentially anionic groups are in fact in the anionic state. To obtain such a ratio, the calculation shall take into account all negative charges in the anionic substance and shall then adjust the quantity of transfecting compound, and eventually of cationic compound, necessary to obtain the desired theoretical charge ratio indicated above. The quantities and the concentrations of the other ingredients shall be adjusted in function of their respective molar masses and their number of positive charges. The ratio is not specifically limited: quantities are selected so that the ratio between the number of positive charges in the cationic lipid and the number of negative charges in the anionic substance is between 0.05 and 20, preferably between 2.5 and 15, and most preferably around 2.5 to 10. Furthermore, the concentration of the negatively-charged anionic substance, which may be added to the compound of the invention to form said complexes of the invention may range from 10 μg/ml to 10000 μg/ml. In a preferred embodiment of the invention, the concentration of anionic substance ranges from 100 μg/ml to 1000 μg/ml.

The invention is also directed to a process for the preparation of the above described complex, comprising the following steps:
contacting at least one compound of the invention with at least one substance of interest,
and recovering said complex, optionally after a purification or selection step.

In a preferred embodiment, at least part of the compound contacted with said substance of interest is substituted, preferably with a ligand able to recognise and to bind specifically with a cell membrane receptor, preferably with an adrenoceptor, and more preferably with a beta1-adrenoceptor.

Where the complex of the invention further comprises:
(c) at least one substituting moiety; and/or
(d) at least one targeting component; and/or
(e) at least one peptide which is capable of causing membrane disruption; and/or
(f) at least one cationic compound selected from the group consisting of cationic lipids and cationic polymers; and/or
(g) at least one colipid.
said process comprises the steps of:
   first mixing said compound (a) with said additional element (c) and/or (d) and/or (e) and/or (f) and/or (g) and then adding the anionic substance of interest (b) in order to form complexes, or
   first complexing said compound (a) with the anionic substance of interest (b) and then mixing the formed complex with said additional element (c) and/or (d) and/or (e) and/or (f) and/or (g).

The process can further comprise a sizing procedure. Methods which can be used in this sizing step include, but are not limited to, extrusion, sonication and microfluidization, size exclusion chromatography, field flow fractionation, electrophoresis and ultracentrifugation.

The invention also encompasses a composition, preferably for transferring an substance of interest into a cell and or tissue, wherein said composition comprises at least one complex or compound of the invention as previously disclosed. Said composition is particularly useful for the delivery of nucleic acids to cells or tissues of a subject in connection with nucleic acid transfer based therapy methods but are not limited to such uses. The term “gene therapy method or vaccine therapy” is preferably understood as a method for the introduction of a nucleic acid into cells either in vivo or by introduction into cells in vitro followed by re-implantation into a subject. “Gene therapy” in particular concerns the case where the gene product is expressed in a tissue as well as the case where the gene product is excreted, especially into the blood stream. The introduction or transfer process of an anionic substance of interest into a cell is by itself well known. “Introduction or transfer” means that the substance is transferred into the cell and is located, at the end of the process, inside said cell or within or on its membrane. If the substance is a nucleic acid, “introduction or transfer” is also referred to as “transfection”. Transfection can be verified by any appropriate method, for example by measuring the expression of a gene encoded by said nucleic acid or by measuring the concentration of the expressed protein or its mRNA, or by measuring its biological effect.

In a preferred embodiment, the composition comprises at least part of the compound or/and complex of the present invention wherein said compound or/and complex is substituted, preferably with a ligand able to recognise and to bind specifically with a cell membrane receptor, preferably with an adrenoceptor, and more preferably with a beta1-adrenoceptor.

This composition of the present invention can be formulated in various forms, e.g. in solid, liquid, powder, aqueous, lyophilized form. In a preferred embodiment, this composition further comprises a pharmaceutically acceptable carrier, allowing its use in a method for the therapeutic treatment of humans or animals. In this particular case, the carrier is preferably a pharmaceutically suitable injectable carrier or diluent (for examples, see Remington's Pharmaceutical Sciences, 16^{th} ed. 1980, Mack Publishing Co). Such a carrier or diluent is pharmaceutically acceptable, i.e. is non-toxic to a recipient at the dosage and concentration employed. It is preferably isotonic, hypotonic or weakly hypertonic and has a relatively low ionic strength, such as provided by a sucrose solution. Furthermore, it may contain any relevant solvents, aqueous or partly aqueous liquid carriers comprising sterile, pyrogen-free water, dispersion media, coatings, and equivalents, or diluents (e.g. Tris-HCl, acetate, phosphate), emulsifiers, solubilizers or adjuvants. The pH of the pharmaceutical preparation is suitably adjusted and buffered in order to be useful in in vivo applications. It may be prepared either as a liquid solution or in a solid form (e.g. lyophilized) which can be suspended in a solution prior to administration. Representative examples of carriers or diluents for an injectable composition include water, isotonic saline solutions which are preferably buffered at a physiological pH (such as phosphate buffered saline or Tris buffered saline), mannitol, dextrose, glycerol and ethanol, as well as polypeptides or proteins such as human serum albumin. For example, such composition comprise 10 mg/ml mannitol, 1 mg/ml HSA, 20 mM Tris pH 7.2 and 150 mM NaCl.

The invention more particularly relates to a composition comprising at least one of the complexes described above and at least one adjuvant capable of improving the transfection capacity of said complex. Adjuvants may be selected from the group consisting of a chloroquine, protic polar compounds, such as propylene glycol, polyethylene glycol, glycerol, EtOH, 1-methyl L-2-pyrrolidone or their derivatives, or aprotic polar compounds such as dimethylsulfoxide (DMSO), diethylsulfoxide, di-n-propylsulfoxide, dimethylsulfone, sulfolane, dimethylformamide, dimethylacetamide, tetramethylurea, acetonitrile or their derivatives.

The composition of the present invention can be administered into a vertebrate tissue, locally and/or systematically. This administration may be carried out by an intradermal, subdermal, intravenous, intramuscular, intranasal, intracerebral, intratracheal, intraarterial, intraperitoneal, intravesical, intrapleural, intracoronary or intratumoral injection, by means of a syringe or other devices. Transdermal administration is also contemplated, such as inhalation, aerosol routes, instillation or topical application. “Vertebrate” as used herein is intended to have the same meaning as commonly understood by one of ordinary skill in the art. Particularly, “vertebrate” encompasses mammals, and more particularly humans.

According to the present invention, the composition can be administered into tissues of the vertebrate body including those of muscle, skin, brain, lung, liver, spleen, bone marrow, thymus, heart, lymph, bone, cartilage, pancreas, kidney, gall bladder, stomach, intestine, testis, ovary, uterus, rectum, nervous system, eye, gland, connective tissue, blood, tumor, etc.

Applied to in vivo nucleic acid transfer therapy, this invention allows repeated administration to the patient without risk of the administered preparation to induce a significant immune reaction. Additionally, the invention greatly limit the spread-off of the complex throughout the body, and non-specific transfer of the substance of interest into non desirable cells and/or tissues. Administration may be by single or repeated dose, once or several times after a certain period of time. Repeated administration allows a reduction of the dose of active substance, in particular DNA, administered at a single time. The route of administration and the appropriate dose varies depending on several parameters, for example the individual patient, the disease being treated, or the nucleic acid being transferred.

According to the invention, “cells” include prokaryotic cells and eukaryotic cells, yeast cells, plant cells, human or animal cells, in particular mammalian cells. In particular, cancer cells should be mentioned. The invention can be applied in vivo to the interstitial or luminal space of tissues in the lungs, the trachea, the skin, the muscles, the brain, the liver, the heart, the spleen, the bone marrow, the thymus, the bladder, the lymphatic system, the blood, the pancreas, the stomach, the kidneys, the ovaries, the testicles, the rectum, the peripheral or central nervous system, the eyes, the lymphoid organs, the cartilage, or the endothelium. In preferred embodiments, the cell will be a muscle cell, as stem cell of the hematopoietic system or an airways cell, more especially a tracheal or pulmonary cell, and preferably a cell of the respiratory epithelium.

The present invention also encompasses a process for transferring a nucleic acid into cells or tissues wherein said process comprises contacting said cells or said tissues with at least one complex or composition according to the invention. This process may be applied by direct administration of said complex or composition to cells or tissues of the animal in vivo, or by in vitro treatment of cells which were recovered from the animal and then re-introduced into the animal body (ex vivo process). In in vitro applications, cells cultivated on an appropriate medium are placed in contact with a suspension containing a complex or composition of the invention. After an incubation time, the cells are washed and recovered. Introduction of the active substance can be verified (eventually after lysis of the cells) by any appropriate method.

In the case of in vivo treatment according to the invention, in order to improve the transfection rate, the patient may undergo a macrophage depletion treatment prior to administration of the pharmaceutical preparation as described above. Such a technique is described in the literature (refer particularly to Van Rooijen et al., 1997, TibTech, 15, 178-184).

Finally, the present invention also provides the use of a compound or complex according to the invention for the preparation of a pharmaceutical composition for curative, preventive or vaccine treatment of mammals. Preferably, such compositions are intended for nucleic acid transfer and more preferably for the treatment of the human or animal body by gene therapy. Within the meaning of the present invention, “gene therapy” has to be understood as a method for introducing any therapeutic gene into a cell. Thus, it also includes immunotherapy that relates to the introduction of a potentially antigenic epitope into a cell to induce an immune response which can be cellular or humoral or both. “Treatment” as used herein refers to prophylaxis and therapy. It concerns both the treatment of humans and animals. A “therapeutically effective amount of a compound, complex or a composition” is a dose sufficient for the alleviation of one or more symptoms normally associated with the disease desired to be treated. A method according to the invention is preferentially intended for the treatment of the diseases listed above.

The invention further concerns the use of a compound or of a complex as defined above for the preparation of a composition for curative, preventive or vaccine treatment of man or animals, preferably mammals, and more specifically for gene therapy use.

The invention further concerns the use of a compound of the invention for the preparation of a complex for transferring an substance of interest into a cell.

As previously indicated, the present invention extends to the use of ligands, or derivatives, able to recognise and react with adrenoceptors, especially beta1 adrenoceptors, for targeting vectors towards cells and/or tissues expressing such membrane receptors. Said expression can be natural, for example in the case of myocardiocytes or neural cells (see above) or artificial when said expression is directed by genetic modification of the targeted cells. According to said special embodiment, vector should be understood as designating plasmid, non-viral and viral vectors, as those previously disclosed. With this respect, one viral vector which is particularly appropriate is an adenoviral vector (for a review see for example Hitt et al. Advances in Pharmacology 40 (1997) 137-206). Preferably, it is replication-defective, especially for E1 functions by total or partial deletion of the respective region. Additionally, the adenoviral backbone of the vector may comprise additional modifications, such as deletions, insertions or mutations in one or more viral genes (see WO 94/28152, WO97/04119 EP98401722.8). In addition, adenoviral virions or empty adenoviral capsids can also be used to transfer nucleic acids (i.e. plasmidic vectors) by a virus-mediated cointernalization process as described in U.S. Pat. No. 5,928,944. This process can be accomplished in the presence of a compound of the present invention.

Adeno associated virus (AAV) vectors can also be used which combines non pathogenicity, broad tropism and infectivity, and long term persistence. In the context of the invention, an adeno associated viral vector may derived from all the AAV serotypes. The preparation of AAV vectors is available in the art (see for example, viral vectors: basic science and gene therapy. (2000) 11-96. Cid-Arregui and Garcia-Carranca ed. Eaton Publishing.).

A retroviral vector is also suitable. Retroviruses are a class of integrative viruses which replicate using a virus-encoded reverse transcriptase, to replicate the viral RNA genome into double stranded DNA which is integrated into chromosomal DNA of the infected cells. The numerous vectors described in the literature may be used within the framework of the present invention and especially those derived from murine leukemia viruses, especially Moloney (Gilboa et al., 1988, Adv. Exp. Med. Biol. 241, 29) or Friend's FB29 strains (WO95/01447).

Finally, poxviral vectors are a group of complex enveloped viruses that distinguish from the above-mentioned viruses by their large DNA genome and their cytoplasmic site of replication. Preferred poxviral vector are vaccinia viruses, such as for example the Copenhagen strain (Goebel et al., 1990, Virol. 179, 247-266 and 517-563), the Wyeth strain and the modified Ankara (MVA) strain (Antoine et al., 1998, Virol. 244, 365-396).

These and other embodiments are disclosed or are obvious from and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on Internet, e.g. under http://www.ncbi.nlm.nih.gov/PubMed/medline.html. Further databases and addresses, such as http://www.ncbi.nlm.nih.gov, http://www.infobiogen.fr, http://www.fmi.ch/biology/research_tools.html, http://www.tigr.org, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The methods, compositions and uses of the invention can be applied in the treatment of all kinds of diseases the treatment and/or diagnostic of which is related to or dependent on the transfer of nucleic acids in cells. The compositions, and uses of the present invention may be desirably employed in humans, although animal treatment is also encompassed by the uses described herein.

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced different from what is specifically described herein.

The disclosure of all patents, publications published patent applications, and database entries cited in the present application are hereby incorporated by reference in their entirety to the same extend as if each such individual patent, publication and database entry were specifically and individually indicated to be incorporated by reference and were set forth in its entirety herein.

LEGENDS

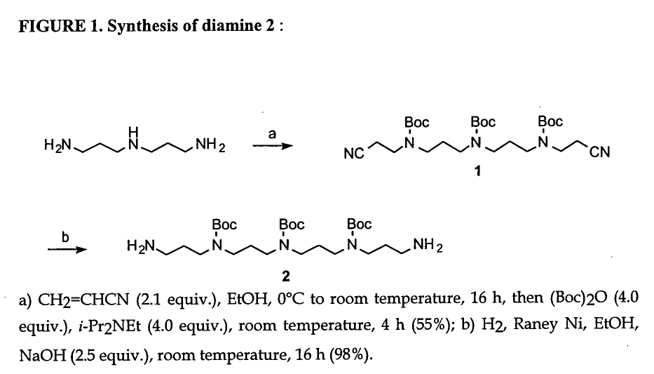

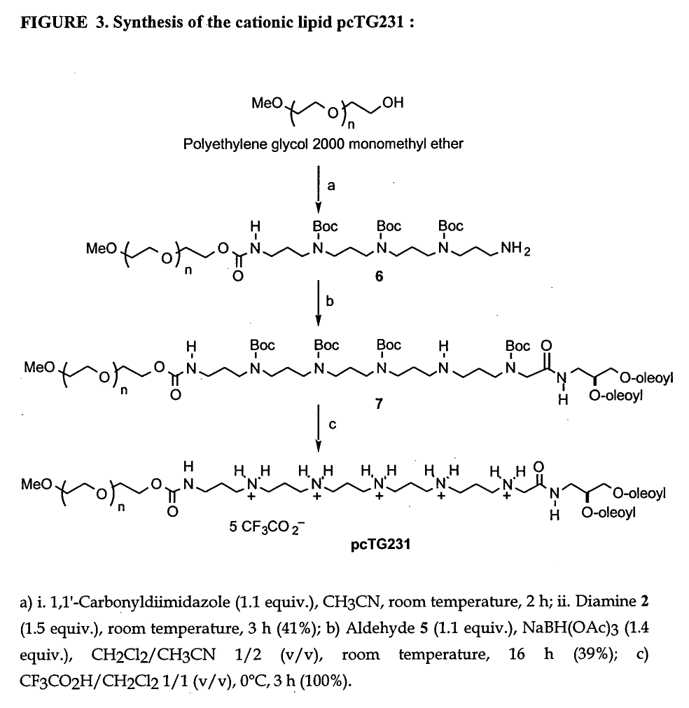

FIGS. 1-3: Illustrate pcTG231 and pcTG238 synthesis.

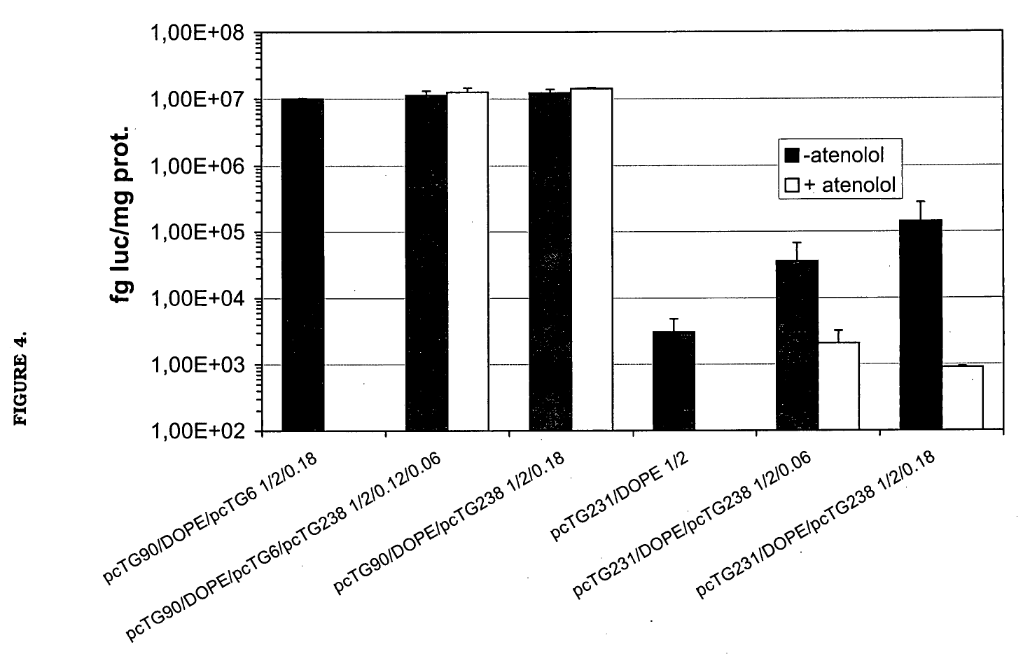

FIG. 4 shows the expression of the luciferase gene by cells expressing an adrenoceptor transfected by cationic lipid/nucleic acid complexes comprising a transfecting compound according to the invention (pcTG231) and/or a targeting compound according to the invention (pcTG238). The transfection procedure has been done in the presence or absence of atenolol as a competitor for beta1-adrenoceptor binding.

FIG. 5 is a targeting component (d) of formula X (i.e. pcTG238 ) in which p is a positive integer from 4 to 220, preferably from 22 to 110 and more preferably about 44 (see page 27 of the specification).

### EXAMPLES

1. Synthesis of a Compound According to the Invention (pcTG231) (see FIGS. 1-3):

1.1. Synthesis of Dinitrile 1

A solution of acrylonitrile (1.06 ml, 16.13 mmol) in ethanol (16 ml) was added dropwise to an ice-cold solution of dipropylenetriamine (1.01 g, 7.68 mmol) in ethanol (77 ml). The mixture was stirred overnight at room temperature. N,N-Diisopropylethylamine (3.97 g, 30.73 mmol) and di-tert-butyl dicarbonate (6.71 g, 30.73 mmol) dissolved in tetrahydrofurane (THF, 16 ml) were then added. The reaction medium was stirred for 4 h at room temperature, concentrated in vacuo, and purified by chromatography on a silica gel column (ether/hexane 60/40, then 75/25) to give 2.29 g (55%) of dinitrile 1 as a colorless oil. ¹H NMR (200 MHz, CDCl₃): δ 3.46 (t, J=6.5 Hz, 4H, >N—CH₂—CH₂—CN), 3.30-3.00 (m, 8H, —CH₂—N(Boc)-), 2.59 (m, 4H, —CH₂—CN), 1.72 (m, 4H, >N—CH₂—CH₂—CH₂—N<), 1.46 and 1.45 (2 s, 27H, t-Bu-).

1.2. Synthesis of Diamine 2:

A solution of dinitrile 1 (2.29 g, 4.26 mmol) in ethanol (20 ml) and water (2 ml) was made basic by addition of sodium hydroxide (0.43 g, 10.7 mmol) and was hydrogenated on Raney nickel (0.54 g) for 16 h at room temperature. The reaction was filtered on celite, the catalyst was washed with methanol (2×20 ml), and the filtrate was concentrated in vacuo. Then water (20 ml) was added and the mixture was extracted with ether (50 ml), dried over Na₂SO₄, concentrated in vacuo, and chromatographed on a silica gel column (CH₂Cl₂/MeOH/NH₄OH 55/40/5) to give 2.28 g (98%) of diamine 2 as a colorless oil. ¹H NMR (200 MHz, CDCl₃): δ 3.35-3.10 (m, 12H, —CH₂—N(Boc)-); 2.68 (t, J=6.7 Hz, 4H, —CH₂—NH₂); 1.85-1.50 (m, 8H, >N—CH₂—CH₂—CH₂—N<); 1.45 (s, 27H, t-Bu-).

1.3. Synthesis of Bromoacetamide 3 (Nazih et al., 2000, D. Synlett, 635)

A—Synthesis of (S)-3-[(N-tert-butoxycarbonyl)amino]propane-1,2-diol A

A solution of ditertiobutyl dicarbonate (0.958 g, 4.39 mmol) in tetrahydrofuran (THF, 2 ml) was added to a solution of (S)-3-aminopropane-1,2-diol (0.200 g, 2.19 mmol) and of diisopropylethylamine (0.567 g, 4.39 mmol) in THF/water (5/1, v/v, 6 ml). After stirring for 3 h at room temperature, the medium was acidified (pH 3) with 10% aqueous HCl and extracted twice with ether (30 ml). The combined organic phases were washed with brine, dried over sodium sulfate, concentrated in vacuo, and chromatographed on a silica gel column (eluent: ethyl acetate) to give diol A (0.408 g; 97%) as a colorless liquid. 1H NMR (200 MHz, CDCl3): δ 4.96 (m, 1H, —CO—NH—); 3.73 (quint., J=5.0 Hz, 1H, >CH—O—); 3.58 (m, 2H, —CH2-O—); 3.27 (m, 2H, —NH—CH2-); 1.45 (s, 9H, t-Bu-).

B. Synthesis Diester 2′

Dicyclohexylcarbodiimide (1.10 g, 5.33 mmol) in dichloromethane (2 ml) was added to a solution of diol A (0.408 g, 2.13 mmol), oleic acid (1.51 g, 5.33 mmol) and 4-(dimethylamino)pyridine (0.026 g, 0.21 mmol) in dichloromethane (20 ml). After stirring for 16 h at room temperature, the precipitated dicyclohexylurea was removed by filtration and the filtrate was concentrated in vacuo and chromatographed on a silica gel column (eluent: dichloromethane) to give the diester 2′ (1.28 g; 83%) as a colorless oil. 1H NMR (200 MHz, CDCl3): δ 5.34 (m, 4H, —CH═); 5.09 (quint., Japp=5.3 Hz, 1H, >CH—O—); 4.73 (m, 1H, —CO—NH—); 4.27 and 4.11 (2 dd, J=12.0, 4.2, 5.7 Hz, 2H, —CH2-O—); 3.35 (m, 2H, —NH—CH2-); 2.31 (t, J=7.4 Hz, 4H, —O—C(O)—CH2-); 2.01 (m, 8H, —CH2-CH═); 1.61 (m, 4H, —O—C(O)—CH2-CH2-); 1.44 (s, 9H, t-Bu-); 1.30 and 1.27 (2 br s, 40H, —CH2-); 0.88 (t, J=6.4 Hz, 6H, Me-).

C—Synthesis of Bromoacetamide 3:

Bromoacetyl bromide (0.53 ml, 6.11 mmol) was added to a solution of diester 2′ (1.01 g, 1.53 mmol) and of methanol (0.13 ml, 3.06 mmol) in dichloromethane (15 ml). After stirring for 20 min. at room temperature, potassium carbonate (2.20 g, 15.9 mmol) was added and the acylation was allowed to proceed until complete (Thin-layer chromatography monitoring; ca. 1 h). The suspension was then filtered on celite”. The filtrate was concentrated in vacuo and chromatographed on a silica gel column (eluent: ether/hexane 30/70 to 40/60) to give bromoacetamide 3 (1.07 g; 94%) as a colorless oil. 1H NMR (200 MHz, CDCl3): δ 6.81 (m, 1H, —CO—NH—); 5.34 (m, 4H, —CH═); 5.14 (m, 1H, >CH—O—); 4.28 and 4.13 (2 dd, J=12.0, 4.4, 5.4 Hz, 2H, —CH2-O—); 3.88 (s, 2H, Br—CH2-); 3.53 (m, 2H, —NH—CH2-); 2.34 and 2.33 (2 t, J=7.5 Hz, 4H, —O—C(O)—CH2-); 2.01 (m, 8H, —CH2-CH═); 1.62 (m, 4H, —O—C(O)—CH2-CH2-); 1.30 and 1.27 (2 br s, 40H, —CH2-); 0.88 (t, J=6.4 Hz, 6H, Me-).

1.4. Synthesis of Alcohol 4:

A solution of bromoacetamide 3 (350 mg, 0.472 mmol) in chloroform (1 ml) was added to a solution of 1-amino-3-propanol (71 mg, 0.94 mmol) in acetonitrile (5 ml). After stirring for 24 h at room temperature, the mixture was diluted with ether (30 ml), washed with water, dried over Na₂SO₄, and concentrated in vacuo. The residue was dissolved in THF (8 ml) and a solution of N,N-diisopropylethylamine (61 mg, 0.47 mmol) in THF (1 ml) was added followed by a solution of di-tert-butyl dicarbonate (67 mg, 0.57 mmol) in THF (1 ml). The solution was stirred for 3 h at room temperature, concentrated in vacuo, and chomatographed on a silica gel column (eluent: ethanol) to give 332 mg (84%) of alcohol 4 as a colorless oil. ¹H NMR (200 MHz, CDCl₃—CD₃CO₂D): δ 5.34 (m, 4H, —CH═); 5.11 (m, 1H, >CH—O—C(O)—); 4.26 and 4.10 (2 dd, J=12.0, 5.9, 4.1 Hz, 2H, —CH₂—O—C(O)—); 3.86 (s, 2H, —N(Boc)-CH₂—C(O)—); 3.66 (t, J=5.6 Hz, 2H, HO—CH₂—); 3.47 (m, 4H, —CH₂—N(Boc)- and —C(O)—NH—CH₂—); 2.31 (t, J=7.6 Hz, 4H, —O—C(O)—CH₂—); 2.01 (m, 8H, —CH₂—CH═); 1.74 (m, 2H, —CH₂—CH₂—N(Boc)-); 1.60 (m, 4H, —O—C(O)—CH₂—CH₂—); 1.45 (s, 9 H, t-Bu-); 1.29 and 1.27 (2 br s, 40H, —CH₂—); 0.88 (t, J=6.4 Hz, 6H, Me-). Elemental analysis. Calculated for C₄₉H₉₀N₂O₈: C, 70.46; H, 10.86; N, 3.35. Found: C, 70.0; H, 11.0; N, 3.3.

1.5. Synthesis of Aldehyde 5:

Phosphorus pentoxide (164 mg, 1.15 mmol) was added to an ice-cold solution of alcohol 4 (320 mg, 0.383 mmol) and of DMSO (0.068 ml, 0.96 mmol) in CH₂Cl₂ (4 ml), and the mixture was vigorously stirred for 2 h at room temperature. After addition of N,N-diisopropylethylamine (198 mg, 1.53 mmol) at 0° C., the mixture was left for an additional 2 h at room temperature. It was then quenched with water (20 ml) and extracted with CH₂Cl₂ (2×25 ml). The combined organic layers were dried over Na₂SO₄ and concentrated in vacuo. The crude aldehyde 5 obtained as a yellow oil was used in the following step without further purification. ¹H NMR (200 MHz, CDCl₃): δ 9.79 (s, 1H, —CHO), 5.34 (m, 4H, —CH═); 5.09 (m, 1H, >CH—O—C(O)—); 4.25 and 4.11 (2 dd, J=11.8, 5.6, 4.3 Hz, 2H, —CH₂—O—C(O)—); 3.85 (s, 2H, —N(Boc)-CH₂—C(O)—NH—); 3.70-3.30 (m, 4H, —CH₂—N(Boc)- and —C(O)—NH—CH₂—); 2.75 (t, J=6.3 Hz, 2H, OHC—CH₂—); 2.31 (t, J=7.5 Hz, 4H, —O—C(O)—CH₂—); 2.00 (m, 8H, —CH₂—CH═); 1.59 (m, 4H, —O—C(O)—CH₂—CH₂—); 1.45 (s, 9H, t-Bu-); 1.29 and 1.27 (2 br s, 40H, —CH₂—); 0.88 (t, J=6.4 Hz, 6H, Me-).

1.6. Synthesis of Amine 6

A solution of polyethylene glycol 2000 monomethyl ether (1.50 g) and of 1,1′-carbonyldiimidazole (134 mg, 0.825 mmol) in acetonitrile (8 ml) was stirred for 2 h at room temperature. Then diamine 2 (614 mg, 1.12 mmol) diluted in acetonitrile (2 ml) was added and the mixture was left for an additional 3 h at room temperature. The solution was concentrated in vacuo and chromatographed on a silica gel column (MeOH/CH₂Cl₂: 5/95 to 20/80) to give 787 mg (41%) of compound 6 as a white powder. ¹H NMR (200 MHz, CDCl₃): δ 4.19 (m, 2H, —CH₂—O—C(O)—NH—); 3.98 (t, J=4.9 Hz, 2H, —O—CH₂—CH₂—O—C(O)—NH—); 3.64 (s, —O—CH₂—CH₂—O—); 3.36 (s, 3H, Me-O—); 3.30-3.05 (m, 14H, —O—C(O)—NH—CH₂— and —N(Boc)-CH₂—); 2.68 (t, J=6.8 Hz, 2H, —CH₂—NH₂); 1.85-1.55 (m, 8H, >N—CH₂—CH₂—CH₂—N<); 1.44 (s, 27H, t-Bu-).

1.7 Synthesis of Diester 7:

A mixture of amine 6 (280 mg, 0.109 mmol) and of aldehyde 5 (100 mg, 0.12 mmol) in CH₃CN/CH₂Cl₂ 2/1 (3 ml) was stirred in the presence of NaBH(OAc)₃ (32 mg, 0.15 mmol) for 16 h at room temperature. The mixture was hydrolyzed with saturated aqueous NaHCO₃ (10 ml) and extracted with CH₂Cl₂ (2×20 ml). The combined organic layers were dried over Na₂SO₄, concentrated in vacuo, and chromatographed on a silica gel column (MeOH/CH₂Cl₂ 5/95 to 10/90) to give 146 mg (39%) of diester 7. ¹H NMR (200 MHz, CDCl₃): δ 5.33 (m, 4H, —CH═); 5.07 (m, 1H, >CH—O—C(O)—); 4.19 (m, 2H, —CH₂—O—C(O)—NH—); 4.24 and 4.09 (2 dd, J=12.2, 5.8, 4.3 Hz, 2H, —CH₂—O—C(O)—); 3.97 (t, J=4.9 Hz, 2H, —O—CH₂—CH₂—O—C(O)—NH—); 3.83 (br s, 2H, —N(Boc)-CH₂—C(O)—NH—); 3.64 (s, —O—CH₂—CH₂—O—); 3.45 (m, 2H, —C(O)—NH—CH₂—); 3.37 (s, 3H, MeO—); 3.32-3.00 (m, 16H, —O—C(O)—NH—CH₂— and —CH₂—N(Boc)-); 2.62 (m, 4H, —CH₂—NH—); 2.30 (t, J=7.5 Hz, 4H, —O—C(O)—CH₂—); 2.00 (m, 8H, —CH₂—CH═); 1.88-1.50 (m, 14H, >N—CH₂—CH₂—CH₂—N< and —O—C(O)—CH₂—CH₂—); 1.44 (s, 36H, t-Bu-); 1.28 and 1.26 (2 br s, 40H, —CH₂—); 0.87 (t, J=6.4 Hz, 6H, Me-).

1.8. Synthesis of the pcTG231

A solution of diester 7 (110 mg, 0.032 mmol) in CH₂Cl₂ (2 ml) was added to a 1/1 mixture of trifluoroacetic acid and of CH₂Cl₂ (4 ml). After stirring for 3 h at 0ᵢC, hexane (10 ml) was added and the mixture was concentrated in vacuo. The so-obtained solid was left in vacuo for 1 h; then it was suspended (vortex) in distilled ether and filtered to give a white powder which was washed with ether and dried in vacuo to give 113 mg (99%) of the cationic lipid pcTG 231. ¹H NMR (200 MHz, CDCl₃—CF₃CO₂D): δ 5.33 (m, 4H, —CH═); 5.20 (m, 1H, >CH—O—C(O)—); 4.40-3.90 (m, 6H, —CH₂—O—C(O)—NH—, —CH₂—O—C(O)— and —O—CH₂—CH₂—O—C(O)—NH—); 4.03 (m, 2H, —NH₂⁺—CH₂—C(O)—NH—); 3.71 (s, —O—CH₂—CH₂—O—); 3.47 (s, 3H, MeO—); 3.40-2.90 (m, 20H, —O—C(O)—NH—CH₂— and —CH₂—NH₂⁺—); 2.40-2.10 (m, 14H, —O—C(O)—CH₂— and —H₂N⁺—CH₂—CH₂—CH₂—NH₂⁺—); 2.00 (m, 8H, —CH₂—CH═); 1.57 (m, 4H, —O—C(O)—CH₂—CH₂—); 1.28 and 1.26 (2 br s, 40H, —CH₂—); 0.87 (t, J=6.4 Hz, 6H, Me-).

2. Synthesis of the Beta-Adrenergic Ligand-Based Lipid pcTG238.

Abreviations. Boc: t-butyloxycarbonyl; Cbz: benzyloxycarbonyl; DCC: 1,3-dicyclohexylcarbodiimide; DMAP: 4-(dimethylamino)pyridine; DMF: dimethylformamide; HOBt: 1-hydroxybenzotriazole; PEG: poly(ethylene glycol); THF: tetrahydrofuran.

Experimental Procedure

The starting compounds 1 and 6 have been described earlier:

Cbz-Tyramine 1: Rocchiccioli, F.; Jarreau, F. -X.; Pais, M. Tetrahedron 1978, 34, 2917-2926 2-(Trimethylsilyl)ethyl hemisuccinate 6: Pouzar, V.; Drasar, P.; Cerny, I.; Havel, M. Synth. Commun. 1984, 14, 501-505

Epoxyde 2

Reaction According to Schlecker, R.; Thieme, P. C. Tetrahedron 1988, 44, 3289-3294.

A solution of diethyl azodicarboxylate (382 mg, 2.19 mmol) in THF (2 ml) was added to a solution of Cbz-tyramine 1 (567 mg, 2.09 mmol), (R)-glycidol (163 mg, 2.19 mmol), and triphenylphosphine (575 mg, 2.19 mmol) in THF (10 ml) at 0° C. After stirring for 16 h at room temperature, the solution was concentrated in vacuo and the residue was triturated with ether (10 ml), filtered, and washed with ether. The filtrate was concentrated and chromatographed on a silica gel column (eluent: CH₂Cl₂ then CH₂Cl₂/Et₂O 98/2) to give 469 mg (69%) of epoxyde 2 as a white solid. Mp: 99-100° C. ¹H NMR (200 MHz, CDCl₃): δ 7.34 (s, 5H, -Ph); 7.09 and 6.86 (2 d, J=8.6 Hz, 4H, —C₆H₄—); 5.09 (s, 2H, —CH₂-Ph); 4.73 (m, 1H, —NH-Cbz); 4.20 and 3.94 (2 dd, J=11.0, 3.2, 5.6 Hz, 2H, —C₆H₄—O—CH₂—); 3.50-3.30 (m, 3H, —CH₂—NH-Cbz, >CH—O—); 2.90 and 2.76 (apparent t, J=4.5 Hz, and m, 4H, —O—CH₂—, —C₆H₄—CH₂—).

Carbamate 3

A mixture of epoxyde 2 (0.44 g, 1.3 mmol) and of isopropylamine (2.85 ml, 33.4 mmol) in methanol (2 ml) was stirred overnight at room temperature. After concentration in vacuo, the product was dissolved in THF (5 ml) and N,N-diisopropylethylamine (0.17 g, 1.3 mmol) was added, followed by di-tert-butyl dicarbonate (0.35 g, 1.6 mmol). After stirring for 4 h at room temperature, the solution was concentrated in vacuo and chromatographed on a silica gel column (eluent: CH₂Cl₂/Et₂O 98/2 to 95/5) to give 463 mg (71%) of carbamate 3. ¹H NMR (200 MHz, CDCl₃): δ 7.34 (s, 5H, -Ph); 7.09 and 6.84 (2 d, J=8.5 Hz, 4H, —C₆H₄—); 5.09 (s, 2H, —CH₂-Ph); 4.74 (m, 1H, —NH-Cbz); 4.24-3.80 (m, 4H, —C₆H₄—O—CH₂—, >CH—OH, —N(Boc)-CHMe₂); 3.48-3.32 (m, 4H, —CH₂—NH-Cbz, —CH₂—N(Boc)-); 2.75 (br t, J=6.9 Hz, 2H, —C₆H₄—CH₂—); 1.49 (s, 9H, t-Bu); 1.17 and 1.14 (2 d, J=7.1 Hz, 6H, —CHMe₂).

Amine 4

Carbamate 3 (425 mg, 0.87 mmol) in ethanol (10 ml) was hydrogenolysed in the presence of 10% palladium on carbon (43 mg) under 1 atmosphere of hydrogen pressure for 6 h at room temperature. The reaction mixture was filtered on celite® and the filtrate was concentrated in vacuo and chromatographed on a silica gel column (MeOH/CH₂Cl₂ 4/96 to 15/85) to afford 235 mg (76%) of amine 4 as a yellow oil. ¹H NMR (200 MHz, CDCl₃): δ 7.11 and 6.85 (2 d, J=8.5 Hz, 4H, —C₆H₄—); 4.25-3.75 (m, 4H, —C₆H₄—O—CH₂—, >CH—OH, —N(Boc)-CHMe₂); 3.38 (m, 2H, —CH₂—N(Boc)-); 2.95 (t, J=6.8 Hz, 2H, —CH₂—NH₂); 2.71 (t, J=6.9 Hz, 2H, —C₆H₄—CH₂—); 1.48 (s, 9H, t-Bu); 1.17 and 1.14 (2 d, J=7.4 Hz, 6H, —CHME₂).

Acid 5

Amine 4 (217 mg, 0.62 mmol) and succinic anhydride (65 mg, 0.65 mmol) in THF (8 ml) were stirred for 4 h at room temperature. The solution was then concentrated in vacuo and chromatographed on a silica gel column (eluent: MeOH/CH₂Cl₂ 10/90 to 20/80) to give 272 mg (98%) of acid 5 as a yellow sticky oil. ¹H NMR (200 MHz, CDCl₃): δ 7.09 and 6.85 (2 d, J=8.6 Hz, 4H, —C₆H₄—); 5.90 (m, 1H, —NH—C(O)—); 4.27-3.80 (m, 4H, —C₆H₄—O—CH₂—, >CH—OH, —CHMe₂); 3.49 (m, 2H, —CH₂—NH—C(O)—); 3.38 (m, 2H, —CH₂—N(Boc)-); 2.76 (t, J=6.8 Hz, 2H, —CH₂—C₆H₄—); 2.66 and 2.44 (2 m, 4H, —NH—C(O)—CH₂—CH₂—CO₂H); 1.48 (s, 9H, t-Bu); 1.17 and 1.14 (2 d, J=6.8 Hz, 6H, CHMe₂).

2-(Trimethylsilyl)ethyl ester 7

DCC (257 mg, 1.25 mmol) was added to a solution of 2-(trimethylsilyl)ethyl hemisuccinate 6 (249 mg, 1.14 mmol), (S)-1-aminopropane-2,3-diol (156 mg, 1.71 mmol), and HOBt (169 mg, 1.25 mmol) in DMF (2 ml) and the medium was stirred at room temperature for 16 h. DMF was removed by sequential addition of toluene (25 ml) and concentration in vacuo (two times). The residue was taken up in dichloromethane (2×5 ml) and was filtered. Oleic acid (966 mg, 3.42 mmol), DMAP (14 mg, 0.11 mmol), and DCC (706 mg, 3.42 mmol) were added to the filtrate and the mixture was stirred for 16 h at room temperature. It was then filtered again, concentrated in vacuo, and chromatographed on a silica gel column (eluent: Et₂O/hexane 60/40 to 80/20) to give 469 mg (52%) of 2-(trimethylsilyl)ethyl ester 7 as a colorless oil. ¹H NMR (200 MHz, CDCl₃): δ 6.00 (m, 1H, —C(O)—NH—); 5.34 (m, 4H, —CH═); 5.10 (m, 1H, >CH—O—C(O)—); 4.32-4.05 (m, 4H, —CH₂—O—C(O)—); 3.50 (m, 2H, —C(O)—NH—CH₂—); 2.64 and 2.46 (2 m, 4H, —O—C(O)—CH₂—CH₂—C(O)—NH—); 2.33 and 2.31 (2 t, J=7.5, 4H, —O—C(O)—CH₂—); 2.01 (m, 8H, —CH₂—CH═); 1.60 (m, 4H, —O—C(O)—CH₂—CH₂—); 1.30 and 1.27 (2 br s, 40H, —CH₂—); 0.98 (m, 2H, —CH₂—SiMe₃); 0.88 (t, J=6.4 Hz, 6H, Me-); 0.04 (m, 9H, Me3Si—).

Acid 8

1 M Tetrabutylammonium fluoride in THF (1.2 ml, 1.20 mmol) was added to a solution of 2-(trimethylsilyl)ethyl ester 7 (0.49 mg, 0.60 mmol) in THF (6 ml) and was allowed to react for 16 h at room temperature. The reaction mixture was acidified with 10% aqueous HCl and extracted with ether. The ether phase was washed with saturated aqueous NaCl, dried over Na₂SO₄, concentrated in vacuo, and chromatographed on a silica gel column (eluent MeOH/CH₂Cl₂ 2/98 to 10/90) to give 297 mg (69%) of acid 8 as a colorless oil. ¹H NMR (200 MHz, CDCl₃): δ 5.34 (m, 4H, —CH═); 5.10 (m, 1H, >CH—O—C(O)—); 4.27 and 4.12 (2 dd, J=12.1, 5.9, 4.0 Hz, 2H, —CH₂—O—C(O)—); 3.48 (m, 2H, —C(O)—NH—CH₂—); 2.64 and 2.48 (2 m, 4H, HO₂C—CH₂—CH₂—C(O)—NH—); 2.31 (br t, J=8.2 Hz, 2H, —O—C(O)—CH₂—); 2.00 (m, 8H, —CH₂—CH═); 1.60 (m, 4H, —O—C(O)—CH₂—CH₂—); 1.29 and 1.27 (2 br s, 40H, —CH₂—); 0.88 (t, J=6.4 Hz, 6H, Me-).

Amine 9

DCC (79 mg, 0.38 mmol) was added to a solution of acid 8 (250 mg, 0.35 mmol), diamino-PEG [average molecular weight ca. 1000] (520 mg, 0.52 mmol), and HOBt (52 mg, 0.38 mmol) in 1/1 THF/CH₂Cl₂ (6 ml). After stirring for 24 h at room temperature, the precipitated dicyclohexylurea was removed by filtration. The filtrate was concentrated in vacuo and chromatographed on a silica gel column (MeOH/CH₂Cl₂ 3/97 to 10/90) to give 323 mg (54%) of amine 9. ¹H NMR (200 MHz, CDCl₃): δ 5.34 (m, 4H, —CH═); 5.10 (m, 1H, >CH—O—C(O)—); 4.26 and 4.09 (2 dd, J=12.4, 5.7, 4.1 Hz, 2H, —CH₂—O—C(O)—); 3.95-3.25 (m, 8H, —O—CH₂—CH₂—NH—, —C(O)—NH—CH₂—); 3.64 (s, —O—CH₂—CH₂—O—); 3.18 (m, 2H, H₂N—CH₂—); 2.75-2.45 (m, 4H, —NH—C(O)—CH₂—CH₂—C(O)—NH—); 2.32 and 2.30 (2 t, J=7.4 Hz, 4H, —O—C(O)—CH₂—); 2.00 (m, 8H, —CH₂—CH═); 1.60 (m, 4H, —O—C(O)—CH₂—CH₂—); 1.29 and 1.27 (2 br s, 40H, —CH₂—); 0.88 (t, J=6.3 Hz, 6H, Me-).

Lipid 10

DCC (20 mg, 0.096 mmol) was added to a solution of acid 5 (43 mg, 0.096 mmol), amine 9 (150 mg, 0.087 mmol), and HOBt (13 mg, 0.096 mmol) in 1/1 THF/CH₂Cl₂ (2 ml). After stirring for 24 h at room temperature, the precipitated dicyclohexylurea was removed by filtration. The filtrate was concentrated in vacuo, dissolved in chloroform (25 ml) and washed with a mixture of saturated aqueous NaHCO₃ (5 ml) and saturated aqueous NaCl (10 ml). The organic layer was dried with Na₂SO₄, concentrated in vacuo, and chromatographed on a silica gel column (eluent: MeOH/CH₂Cl₂ 3/97 to 8/92) to give 153 mg (82%) of lipid 10 contaminated by a slightly less polar compound which will be removed after the next step. ¹H-NMR (500 MHz, CDCl₃): δ 7.09 and 6.83 (2 d, J=8.5 Hz, 4H, —C₆H₄—); 6.67, 6.55, and 6.22 (3 m, 4H, —NH—C(O)—); 5.33 (m, 4H, —CH═); 5.09 (m, 1H, >CH—O—C(O)—); 4.24 and 4.09 (2 dd, J=12.0, 5.9, 3.8 Hz, 2H, —CH₂—O—C(O)—); 4.08-3.93 (m, 2H, —C₆H₄—O—CH₂—); 3.63 (s, —O—CH₂—CH₂—O—); 3.54 (t, J=5.1 Hz, 4H, —O—CH₂—CH₂—NH—C(O)—); 3.57-3.35 (m, [includes at □ 3.42 a br q, J=5.1 Hz], 10H, —CH₂—NH—C(O)—, —CH₂—N(Boc)-); 2.73 (t, J=7.2 Hz, 2H, —C₆H₄—CH₂—); 2.54-2.44 (m, 8H, —NH—C(O)—CH₂—CH₂—C(O)—NH—); 2.31 and 2.29 (2 t, J=7.6 Hz, 4H, —O—C(O)—CH₂—); 2.00 (m, 8H, —CH₂—CH═); 1.60 (m, 4H, —O—C(O)—CH₂—CH₂—); 1.48 (s, 9H, t-Bu); 1.29 and 1.26 (2 br s, 40H, —CH₂—); 1.17 and 1.13 (2 d, J=6.7Hz, 6H, —CHMe₂); 0.87 (t, J=6.9Hz, 6H, Me-).

Lipid pcTG238

Compound 10 (100 mg, 0.046 mmol) in dichloromethane (1 ml) was treated for 2 h with a 1/1 mixture of trifluoroacetic acid and dry dichloromethane (2 ml) at 0° C. Hexane (5 ml) was then added and the mixture was concentrated in vacuo. The residue was dissolved in chloroform (20 ml) and washed with saturated aqueous NaHCO₃ (10 ml). The aqueous layer was extracted with additionnal chloroform (20 ml) and the combined organic layers were dried with Na₂SO₄, concentrated in vacuo, and chromatographed on a silica gel column (eluent: MeOH/CH₂Cl₂ 8/92 to 15/85) to give 69 mg (73%) of lipid pcTG238. ¹H-NMR (500 MHz, CDCl₃): δ 7.09 and 6.84 (2 d, J=8.5 Hz, 4H, —C₆H₄—); 6.72, 6.64, and 6.34 (3 m, 4H, —NH—C(O)—); 5.34 (m, 4H, —CH═); 5.09 (m, 1H, >CH—O—C(O)—); 4.25 and 4.09 (2 dd, J=11.9, 5.9, 4.0 Hz, 2H, —CH₂—O—C(O)—); 4.15 (m, 1H, >CH—OH); 4.01 and 3.96 (2 dd, J=9.7, 5.4, 5.3 Hz, 2H, —C₆H₄—O—CH₂—); 3.64 (s, —O—CH₂—CH₂—O—); 3.54 (t, J=5.1 Hz, 4H, —O—CH₂—CH₂—NH—C(O)—); 3.51-3.37 (m, 8H, —CH₂—NH—C(O)—); 3.04 and 2.86 (m and dd, J=11.9, 8.3 Hz, 3H, —NH—CH<, —CH₂—NH—); 2.73 (t, J=7.0 Hz, 2H, —C₆H₄—CH₂—); 2.54-2.42 (m, 8H, —NH—C(O)—CH₂—CH₂—C(O)—NH—); 2.32 and 2.30 (2 t, J=7.7 Hz, 4H, —O—C(O)—CH₂—); 2.00 (m, 8H, —CH₂—CH═), 1.60 (m, 4H, —O—C(O)—CH₂CH₂—); 1.29 and 1.26 (2 br s, 40H, —CH₂—), 1.21 (d, J=6.2 Hz, 6H, —CHMe₂); 0.88 (t, J=6.9 Hz, 6H, -Me). MALDI-TOF MS alpha-Cyano-4-hydroxycinnamic acid matrix). The spectrum shows two series of peaks (44 Da intervals in each series). Most abundant series: Major peak at m/z=2108.7; Calculated for [C₁₀₉H₂₀₂N₅O₃₃]⁺: 2109.4 (pcTG238+H⁺; n=23). Least abundant series: Major peak at 2174.7; Calculated for [C₁₁₁H₂₀₅N₅NaO₃₄]⁺: 2175.4 (pcTG238+Na⁺; n=24).

3. Preparation of the Cationic Lipid/Nucleic Acid Complex

Lipids were mixed in chloroform and solvent removal was performed overnight at 45° C. using a Rapidvap vortex evaporator (Labconco, Uniequip, Martinsried, Germany). The resulting lipid films were hydrated with a 5% glucose (w/v) solution (5-15 mg/mL cationic lipids) and sonicated (Bransonic 221 ultrasonic water bath from Branson Ultrasonics Corp., Danbury, Conn., USA) until lipids were entirely resuspended. Small liposomes were formed by sequential extrusion through 400 and 200 nm pore diameter polycarbonate membranes (Nuclepore, Costar, Cambridge, Mass., USA) using a Lipex Biomembranes extruder (Vancouver, Canada). Preformed liposomes were stored at 4° C. under inert atmosphere (argon) until use.

Corresponding complexes were formed by mixing plasmid DNA (comprising a gene coding for luciferase) with cationic liposomes. Plasmid DNA was first diluted in 5% glucose to the desired concentration and complex formation was done by rapid addition of extruded liposome suspension to the plasmid solution (volume of liposomal suspension/volume of plasmid DNA solution≃1/3). All complexes were prepared at a final plasmid concentration of 240 microg/mL and kept for at least an overnight time period at 4° C. before use.

4. Determination of Complexation Efficiency of Cationic Lipids with Plasmid DNA

Cationic lipid/nucleic acid complexes samples (0.2 μg DNA) were loaded onto a 1% agarose gel (90 V, 2 h) in TEA-buffer (40 mM Tris, 0.9 mM EDTA, 5 mM sodium acetate/acetic acid, pH 7.8). DNA bands were stained with ethidium bromide (10 μg/mL). Each sample pretreated with 10% volume of sodium dodecyl sulfate were run in parallel as a negative probe.

Complexes comprising nucleic acid and the transfecting compound according to the invention pcTG231 can not migrate in the agarose gel indicating that the nucleic acid is fully complexed. The same result is obtained with complexes further comprising a colipid (DOPE).

5. Cell Culture and Transfection

Cardiomyocytes which express the beta 1 adrenoceptor have been extracted according to Chien et al (J Clin Invest 75 (1985) 1770-80) and Adams et al (J Biol Chem 271 (1996) 1179-86). The primary culture of cardiomyocytes was prepared from whole hearts of fetal C57BL/6 mice. Animals were killed by decapitation and hearts were minced into 1-mm pieces in balanced salt solution [0.8 mM MgSO4-7H2O, 116 mM NaCl, 1 mM Na2HPO4-H2O, 5.4 mM KCl, 5.5 mM glucose, 20 mM Hepes pH 7.4]. Tissues were dissociated in 0.03% collagenase II (Worthington)-0.06% pancreatin (Life Technologies) in this buffer under mechanical agitation at 37° C. for 20 min. The supernatant was discarded and replaced with fresh protease solution for a second 20-min period. The suspension was adjusted to 20% FCS and dissociation was completed by gentle trituration with a Pasteur pipette. Cells were centrifuged at 250×g for 5 min and the pellet was subjected to preplating for 2×30 min in complete medium [Dulbecco's modified Eagle's medium/Medium 199 (Life Technologies) at 4/1 ratio supplemented with 5% horse serum (Life Technologies), 5% FCS (Hyclone), 2 mM glutamine and 40 μg/ml gentamycin] on non-coated dishes. Non-attached cells were collected and seeded at 7.5×104 cells per well in a 48-well plate coated with 0.1% gelatin and maintained in complete medium in a 5% CO2/95% air containing atmosphere at 37° C. The medium was replaced the next day.

Medium was changed once before transfection by cationic lipid/nucleic acid complexes was performed 24 h to 48 h later in absence of serum at a dose of 1.25 microg DNA/well (0.25 mL). Cells were washed 24 h later and were further cultured for another 24 h with complete medium before luciferase expression determination. Luciferase activity (as RLU/mg) was determined in a luminometer (Microlumat LB96P, Berthold, Evry, France). Light units measured for the substrate solution alone were substracted from all readings. Protein concentration was determined by BCA protein assay (Pierce, Monlucon, France), and luciferase activity was calculated as RLU/min/mg protein. Luciferase substrate solution was from Promega.

The same procedure was performed in the case of competition experiments, except that an excess of free atenolol (10⁻⁵ M) was co-incubated with cationic lipid/nucleic acid complexes.

FIG. 4 shows that cells transfected by complexes comprising a transfecting compound according to the invention (pcTG231) express less luciferase (more than 1000× less) compared to cells transfected by complexes comprising a cationic lipid (pcTG90) without a hydrophilic moiety. This result clearly indicates that complexes comprising a targeting compound according to the invention have a very low non-specific transfection efficiency.

Cells expressing β1-adrenoceptor on their surface which have being transfected with complexes comprising a targeting ligand (pcTG238) and a compound of the invention (pcTG231) express both luciferase hereby illustrating transfer of the luciferase gene into cells. Results further indicate that said luciferase expression is inhibited when the transfection is made in the presence of atenolol (an adrenoceptor antagonist). This result clearly shows that the targeting compound according to the invention allow the targeting of cells expressing an adrenoceptor and that the transfecting compound according to the invention allows the transfection of cells when it is used in combination with a targeting element.

1. A compound comprising:
(i) a polar headgroup spacer,
(ii) at least one hydrophobic moiety, and
(iii) at least one hydrophilic polymer,
wherein said polar headgroup spacer is coupled to said hydrophobic moiety and to said hydrophilic polymer.

2. The compound of claim 1 wherein said polar headgroup spacer is a cationic headgroup spacer.

3. The compound of claim 2 wherein said cationic headgroup spacer comprises from 2 to 7 positive charges.

4. The compound of claim 2 wherein said polar headgroup spacer coupled to said hydrophobic moiety is a cationic lipid.

5. The compound of claim 4 wherein said cationic lipid is of Formula I: 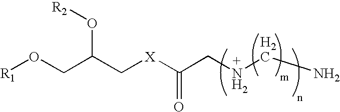
in which:
R₁ and R₂, which are identical or different, are alkyl or alkenyl radicals having 6 to 23 carbon atoms (noted C6-C23), which are linear or branched, or radicals —C(═O)—(C6-C23) alkyl or —C(═O)—(C6-C23) alkenyl, or more particularly —C(═O)—(C12-C20) alkyl or —C(═O)—(C12-C20) alkenyl, which are linear or branched, aryl radicals, cycloalkyl radicals, fluoroalkyl radicals, oxyethylene or oxymethylene groups which are optionally repeated, linear or branched, optionally substituted,
X is an oxygen atom or an amino radical —NR₃, R₃ being a hydrogen atom or an alkyl radical having 1 to 4 carbon atoms,
n is a positive integer from 1 to 6, preferably from 2 to 4,
m is a positive integer from 1 to 6, preferably from 2 to 4, and when n>1, m may be identical or different from said n.

6. The compound of claim 1 wherein said hydrophilic polymer is selected from the group consisting of polyalkylethers, ganglioside Gm1, polyvinylpyrrolidone, polyalkyloxazoline, polyalkylacrylamide, polyalkylacrylate, polyalkylcellulose, polyaspartamide, tetritols, pentitols, hexitols, dulcitol.

7. The compound of claim 1 wherein said hydrophilic polymer is a polyethyleneglycol (PEG).

8. The compound of claim 7 wherein said polyethyleneglycol has a molecular weight ranging between about 1,000 and about 5,000 daltons (Da).

9. The compound of claim 5 which is of formula VII: 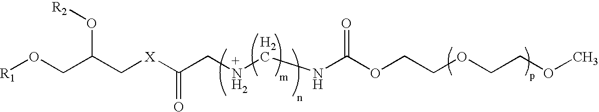
in which Ri, R2, X, n and m are as mentioned in claim 5, p is a positive integer from 4 to 220.

10. The compound of claim 9 which is of formula VIII:

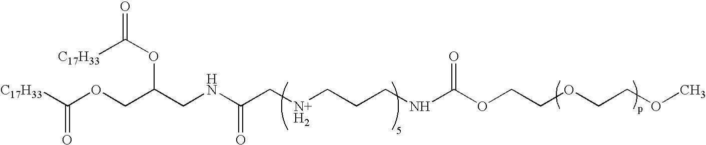

11. A complex comprising (a) at least one compound of claim 1 and (b) at least one substance of interest.

12. The complex of claim 11 wherein said substance of interest is selected from the group consisting of proteins and nucleic acid molecules.

13. The complex of claim 11 which further comprises:
(c) at least one substituting moiety; and/or
(d) at least one targeting component; and/or
(e) at least one peptide which is capable of causing membrane disruption; and/or
(f) at least one cationic compound selected from the group consisting of cationic lipids and cationic polymers; and/or
(g) at least one colipid.

14. A composition comprising at least one compound of claim 1 and a pharmaceutically acceptable carrier therefor.

15. A method for the preparation of a pharmaceutical composition for curative, preventive or vaccine treatment of mammals comprising administering to a patient in need of such treatment an effective amount of the compound according to claim 1.

16. A method for transferring an anionic substance of interest into a cell comprising using an effective amount of the compound according to claim 1.
